SEQUENCE LISTING (379 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 5 aa, Homo sapiens
SYAMS

SEQ ID NO: 3 - protein, 16 aa, Homo sapiens
VTGAVGRTYYPDSVKG

SEQ ID NO: 4 - protein, 8 aa, Homo sapiens
WGDEGFDI

SEQ ID NO: 5 - protein, 11 aa, Homo sapiens
RASQGISNWLA

SEQ ID NO: 6 - protein, 7 aa, Homo sapiens
GASSLQS

SEQ ID NO: 7 - protein, 9 aa, Homo sapiens
QQYSSFPTT

SEQ ID NO: 8 - protein, 7 aa, Homo sapiens
GFTFSSY

SEQ ID NO: 9 - protein, 5 aa, Homo sapiens
GAVGR

SEQ ID NO: 10 - protein, 8 aa, Homo sapiens
WGDEGFDI

SEQ ID NO: 11 - protein, 7 aa, Homo sapiens
SQGISNW

SEQ ID NO: 12 - protein, 3 aa, Homo sapiens
GAS

SEQ ID NO: 13 - protein, 6 aa, Homo sapiens
YSSFPT

SEQ ID NO: 14 - protein, 107 aa, Homo sapiens
DIQMTQSPSSLSASVGDRVTITCRASQGISNWLAWYQQKPGKAPKLLIYGASSLQSGVPSRFSGSGSGTDFTLTISSLQPEDFAVYYCQQYSSFPTTFGQGTKVEIK

SEQ ID NO: 15 - protein, 116 aa, Homo sapiens
QVQLVESGGGLVQPGGSLRLSCAASGFTFSSYAMSWVRQAPGKGLEWVSVTGAVGRTYYPDSVKGRFTISRDNSKNTLYLQMNSLRAEDTAVYYCARWGDEGFDIWGQGTLVTVSS

SEQ ID NO: 16 - DNA, 321 bp, Homo sapiens
gatatccagatgacccagagcccgtctagcctgagcgcgagcgtgggtgatcgtgtgaccattacctgcagagcgagccagggtatttctaattggctggcttggtaccagcagaaaccaggtaaagcaccgaaactattaatttatggtgcttcttctttgcaaagcggggtcccgtcccgttttagcggctctggatccggcactgattttaccctgaccattagcagcctgcaacctgaagactttgcggtttattattgccagcagtattcttcttttcctactacctttggccagggtacgaaagttgaaattaaa

SEQ ID NO: 17 - DNA, 348 bp, Homo sapiens
caggtgcaattggtggaaagcggcggcggcctggtgcaaccgggcggcagcctgcgtctgagctgcgcggcctccggatttacctttagcagctatgcgatgagctgggtgcgccaagcccctgggaagggtctcgagtgggtgagcgttactggtgctgttggtcgtacttattatcctgattctgttaagggtcgttttaccatttcacgtgataattcgaaaaacaccctgtatctgcaaatgaacagcctgcgtgcggaagatacggccgtgtattattgcgcgcgttggggtgatgagggttttgatatttggggccaaggcaccctggtgacggttagctca

SEQ ID NO: 18 - protein, 214 aa, Homo sapiens
DIQMTQSPSSLSASVGDRVTITCRASQGISNWLAWYQQKPGKAPKLLIYGASSLQSGVPSRFSGSGSGTDFTLTISSLQPEDFAVYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 446 aa, Homo sapiens
QVQLVESGGGLVQPGGSLRLSCAASGFTFSSYAMSWVRQAPGKGLEWVSVTGAVGRTYYPDSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 5 aa, Homo sapiens
SYAMS

SEQ ID NO: 21 - protein, 17 aa, Homo sapiens
VISAWGHVKYYADSVKG

SEQ ID NO: 22 - protein, 8 aa, Homo sapiens
WGDEGFDI

SEQ ID NO: 23 - protein, 11 aa, Homo sapiens
RASQGISNWLA

SEQ ID NO: 24 - protein, 7 aa, Homo sapiens
GASSLQS

SEQ ID NO: 25 - protein, 9 aa, Homo sapiens
QQYSSFPTT

SEQ ID NO: 26 - protein, 7 aa, Homo sapiens
GFTFSSY

SEQ ID NO: 27 - protein, 6 aa, Homo sapiens
SAWGHV

SEQ ID NO: 28 - protein, 8 aa, Homo sapiens
WGDEGFDI

SEQ ID NO: 29 - protein, 7 aa, Homo sapiens
SQGISNW

SEQ ID NO: 30 - protein, 3 aa, Homo sapiens
GAS

SEQ ID NO: 31 - protein, 6 aa, Homo sapiens
YSSFPT

SEQ ID NO: 32 - protein, 107 aa, Homo sapiens; identical to SEQ ID NO: 14

SEQ ID NO: 33 - protein, 117 aa, Homo sapiens
QVQLVESGGGLVQPGGSLRLSCAASGFTFSSYAMSWVRQAPGKGLEWVSVISAWGHVKYYADSVKGRFTISRDNSKNTLYLQMNSLRAEDTAVYYCARWGDEGFDIWGQGTLVTVSS

SEQ ID NO: 34 - DNA, 321 bp, Homo sapiens; identical to SEQ ID NO: 16

SEQ ID NO: 35 - DNA, 351 bp, Homo sapiens
caggtgcaattggtggaaagcggcggcggcctggtgcaaccgggcggcagcctgcgtctgagctgcgcggcctccggatttacctttagcagctatgcgatgagctgggtgcgccaagcccctgggaagggtctcgagtgggtgagcgttatttctgcttggggtcatgttaagtattatgctgattctgttaagggtcgttttaccatttcacgtgataattcgaaaaacaccctgtatctgcaaatgaacagcctgcgtgcggaagatacggccgtgtattattgcgcgcgttggggtgatgagggttttgatatttggggccaaggcaccctggtgacggttagctca

SEQ ID NO: 36 - protein, 214 aa, Homo sapiens; identical to SEQ ID NO: 18

SEQ ID NO: 37 - protein, 447 aa, Homo sapiens
QVQLVESGGGLVQPGGSLRLSCAASGFTFSSYAMSWVRQAPGKGLEWVSVISAWGHVKYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 5 aa, Homo sapiens
SYAMS

SEQ ID NO: 39 - protein, 17 aa, Homo sapiens
AINSQGKSTYYADSVKG

SEQ ID NO: 40 - protein, 8 aa, Homo sapiens
WGDEGFDI

SEQ ID NO: 41 - protein, 11 aa, Homo sapiens
RASQGISNWLA

SEQ ID NO: 42 - protein, 7 aa, Homo sapiens
GASSLQS

SEQ ID NO: 43 - protein, 9 aa, Homo sapiens
QQYSSFPTT

SEQ ID NO: 44 - protein, 7 aa, Homo sapiens
GFTFSSY

SEQ ID NO: 45 - protein, 6 aa, Homo sapiens
NSQGKS

SEQ ID NO: 46 - protein, 8 aa, Homo sapiens
WGDEGFDI

SEQ ID NO: 47 - protein, 7 aa, Homo sapiens
SQGISNW

SEQ ID NO: 48 - protein, 3 aa, Homo sapiens
GAS

SEQ ID NO: 49 - protein, 6 aa, Homo sapiens
YSSFPT

SEQ ID NO: 50 - protein, 107 aa, Homo sapiens; identical to SEQ ID NO: 14

SEQ ID NO: 51 - protein, 117 aa, Homo sapiens
QVQLVESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSS

SEQ ID NO: 52 - DNA, 321 bp, Homo sapiens; identical to SEQ ID NO: 16

SEQ ID NO: 53 - DNA, 351 bp, Homo sapiens
caggtgcaattggtggaaagcggcggcggcctggtgcaaccgggcggcagcctgcgtctgagctgcgcggcctccggatttacctttagcagctatgcgatgagctgggtgcgccaagcccctgggaagggtctcgagtgggtgagcgctattaattctcagggtaagtctacttattatgctgattctgttaagggtcgttttaccatttcacgtgataattcgaaaaacaccctgtatctgcaaatgaacagcctgcgtgcggaagatacggccgtgtattattgcgcgcgttggggtgatgagggttttgatatttggggccaaggcaccctggtgacggttagctca

SEQ ID NO: 54 - protein, 214 aa, Homo sapiens; identical to SEQ ID NO: 18

SEQ ID NO: 55 - protein, 447 aa, Homo sapiens
QVQLVESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 5 aa, Homo sapiens
SYAMS

SEQ ID NO: 57 - protein, 17 aa, Homo sapiens
VINPSGNFTNYADSVKG

SEQ ID NO: 58 - protein, 8 aa, Homo sapiens
WGDEGFDI

SEQ ID NO: 59 - protein, 11 aa, Homo sapiens
RASQGISNWLA

SEQ ID NO: 60 - protein, 7 aa, Homo sapiens
GASSLQS

SEQ ID NO: 61 - protein, 9 aa, Homo sapiens
QQYSSFPTT

SEQ ID NO: 62 - protein, 7 aa, Homo sapiens
GFTFSSY

SEQ ID NO: 63 - protein, 6 aa, Homo sapiens
NPSGNF

SEQ ID NO: 64 - protein, 8 aa, Homo sapiens
WGDEGFDI

SEQ ID NO: 65 - protein, 7 aa, Homo sapiens
SQGISNW

SEQ ID NO: 66 - protein, 3 aa, Homo sapiens
GAS

SEQ ID NO: 67 - protein, 6 aa, Homo sapiens
YSSFPT

SEQ ID NO: 68 - protein, 107 aa, Homo sapiens; identical to SEQ ID NO: 14

SEQ ID NO: 69 - protein, 117 aa, Homo sapiens
QVQLVESGGGLVQPGGSLRLSCAASGFTFSSYAMSWVRQAPGKGLEWVSVINPSGNFTNYADSVKGRFTISRDNSKNTLYLQMNSLRAEDTAVYYCARWGDEGFDIWGQGTLVTVSS

SEQ ID NO: 70 - DNA, 321 bp, Homo sapiens; identical to SEQ ID NO: 16

SEQ ID NO: 71 - DNA, 351 bp, Homo sapiens
caggtgcaattggtggaaagcggcggcggcctggtgcaaccgggcggcagcctgcgtctgagctgcgcggcctccggatttacctttagcagctatgcgatgagctgggtgcgccaagcccctgggaagggtctcgagtgggtgagcgttattaatccttctggtaattttactaattatgctgattctgttaagggtcgttttaccatttcacgtgataattcgaaaaacaccctgtatctgcaaatgaacagcctgcgtgcggaagatacggccgtgtattattgcgcgcgttggggtgatgagggttttgatatttggggccaaggcaccctggtgacggttagctca

SEQ ID NO: 72 - protein, 214 aa, Homo sapiens; identical to SEQ ID NO: 18

SEQ ID NO: 73 - protein, 447 aa, Homo sapiens
QVQLVESGGGLVQPGGSLRLSCAASGFTFSSYAMSWVRQAPGKGLEWVSVINPSGNFTNYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 5 aa, Homo sapiens
SYAMS

SEQ ID NO: 75 - protein, 16 aa, Homo sapiens
NTSPIGYTYYAGSVKG

SEQ ID NO: 76 - protein, 8 aa, Homo sapiens
WGDEGFDI

SEQ ID NO: 77 - protein, 11 aa, Homo sapiens
RASQGISNWLA

SEQ ID NO: 78 - protein, 7 aa, Homo sapiens
GASSLQS

SEQ ID NO: 79 - protein, 9 aa, Homo sapiens
QQYSSFPTT

SEQ ID NO: 80 - protein, 7 aa, Homo sapiens
GFTFSSY

SEQ ID NO: 81 - protein, 5 aa, Homo sapiens
SPIGY

SEQ ID NO: 82 - protein, 8 aa, Homo sapiens
WGDEGFDI

SEQ ID NO: 83 - protein, 7 aa, Homo sapiens
SQGISNW

SEQ ID NO: 84 - protein, 3 aa, Homo sapiens
GAS

SEQ ID NO: 85 - protein, 6 aa, Homo sapiens
YSSFPT

SEQ ID NO: 86 - protein, 107 aa, Homo sapiens; identical to SEQ ID NO: 14

SEQ ID NO: 87 - protein, 116 aa, Homo sapiens
QVQLVESGGGLVQPGGSLRLSCAASGFTFSSYAMSWVRQAPGKGLEWVSNTSPIGYTYYAGSVKGRFTISRDNSKNTLYLQMNSLRAEDTAVYYCARWGDEGFDIWGQGTLVTVSS

SEQ ID NO: 88 - DNA, 321 bp, Homo sapiens; identical to SEQ ID NO: 16

SEQ ID NO: 89 - DNA, 348 bp, Homo sapiens
caggtgcaattggtggaaagcggcggcggcctggtgcaaccgggcggcagcctgcgtctgagctgcgcggcctccggatttacctttagcagctatgcgatgagctgggtgcgccaagcccctgggaagggtctcgagtgggtgagcaatacttctcctattggttatacttattatgctggttctgttaagggtcgttttaccatttcacgtgataattcgaaaaacaccctgtatctgcaaatgaacagcctgcgtgcggaagatacggccgtgtattattgcgcgcgttggggtgatgagggttttgatatttggggccaaggcaccctggtgacggttagctca

SEQ ID NO: 90 - protein, 214 aa, Homo sapiens
DIQMTQSPSSLSASVGDRVTITCRASQGISNWLAWYQQKPGKAPKLLIYGASSLQSGVPSRFSGSGSGTDFTLTISSLQPEDFAVYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 91 - protein, 218 aa, Homo sapiens
QVQLVESGGGLVQPGGSLRLSCAASGFTFSSYAMSWVRQAPGKGLEWVSNTSPIGYTYYAGSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 92 - protein, 5 aa, Homo sapiens
SYAMS

SEQ ID NO: 93 - protein, 17 aa, Homo sapiens
VTGAVGRSTYYPDSVKG

SEQ ID NO: 94 - protein, 8 aa, Homo sapiens
WGDEGFDI

SEQ ID NO: 95 - protein, 11 aa, Homo sapiens
RASQGISNWLA

SEQ ID NO: 96 - protein, 7 aa, Homo sapiens
GASSLQS

SEQ ID NO: 97 - protein, 9 aa, Homo sapiens
QQYSSFPTT

SEQ ID NO: 98 - protein, 7 aa, Homo sapiens
GFTFSSY

SEQ ID NO: 99 - protein, 6 aa, Homo sapiens
GAVGRS

SEQ ID NO: 100 - protein, 8 aa, Homo sapiens
WGDEGFDI

SEQ ID NO: 101 - protein, 7 aa, Homo sapiens
SQGISNW

SEQ ID NO: 102 - protein, 3 aa, Homo sapiens
GAS

SEQ ID NO: 103 - protein, 6 aa, Homo sapiens
YSSFPT

SEQ ID NO: 104 - protein, 107 aa, Homo sapiens
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIK

SEQ ID NO: 105 - protein, 117 aa, Homo sapiens
EVQLLESGGGLVQPGGSLRLSCAASGFTFSSYAMSWVRQAPGKGLEWVSVTGAVGRSTYYPDSVKGRFTISRDNSKNTLYLQMNSLRAEDTAVYYCARWGDEGFDIWGQGTLVTVSS

SEQ ID NO: 106 - DNA, 321 bp, Homo sapiens
gatatccagatgacccagagccccagcagcctgagcgccagcgtgggcgacagagtgaccatcacctgtcgggccagccagggcatcagcaactggctggcctggtatcagcagaagcccggcaaggcccccaagctgctgatctacggcgccagctccctgcagagcggcgtgccaagcagattcagcggcagcggctccggcaccgacttcaccctgaccatcagcagcctgcagcccgaggacttcgccacctactactgccagcagtacagcagcttccccaccaccttcggccagggcaccaaggtggaaatcaag

SEQ ID NO: 107 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtgacaggcgccgtgggcagaagcacctactaccccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 108 - protein, 214 aa, Homo sapiens
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 447 aa, Homo sapiens
EVQLLESGGGLVQPGGSLRLSCAASGFTFSSYAMSWVRQAPGKGLEWVSVTGAVGRSTYYPDSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 5 aa, Homo sapiens
SYAMS

SEQ ID NO: 111 - protein, 17 aa, Homo sapiens
VISAWGHVKYYADSVKG

SEQ ID NO: 112 - protein, 8 aa, Homo sapiens
WGDEGFDI

SEQ ID NO: 113 - protein, 11 aa, Homo sapiens
RASQGISNWLA

SEQ ID NO: 114 - protein, 7 aa, Homo sapiens
GASSLQS

SEQ ID NO: 115 - protein, 9 aa, Homo sapiens
QQYSSFPTT

SEQ ID NO: 116 - protein, 7 aa, Homo sapiens
GFTFSSY

SEQ ID NO: 117 - protein, 6 aa, Homo sapiens
SAWGHV

SEQ ID NO: 118 - protein, 8 aa, Homo sapiens
WGDEGFDI

SEQ ID NO: 119 - protein, 7 aa, Homo sapiens
SQGISNW

SEQ ID NO: 120 - protein, 3 aa, Homo sapiens
GAS

SEQ ID NO: 121 - protein, 6 aa, Homo sapiens
YSSFPT

SEQ ID NO: 122 - protein, 107 aa, Homo sapiens; identical to SEQ ID NO: 104

SEQ ID NO: 123 - protein, 117 aa, Homo sapiens
EVQLLESGGGLVQPGGSLRLSCAASGFTFSSYAMSWVRQAPGKGLEWVSVISAWGHVKYYADSVKGRFTISRDNSKNTLYLQMNSLRAEDTAVYYCARWGDEGFDIWGQGTLVTVSS

SEQ ID NO: 124 - DNA, 321 bp, Homo sapiens; identical to SEQ ID NO: 106

SEQ ID NO: 125 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtgatcagcgcctggggccacgtgaagtactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 126 - protein, 214 aa, Homo sapiens; identical to SEQ ID NO: 108

SEQ ID NO: 127 - protein, 447 aa, Homo sapiens
EVQLLESGGGLVQPGGSLRLSCAASGFTFSSYAMSWVRQAPGKGLEWVSVISAWGHVKYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 5 aa, Homo sapiens
SYAMS

SEQ ID NO: 129 - protein, 17 aa, Homo sapiens
AINSQGKSTYYADSVKG

SEQ ID NO: 130 - protein, 8 aa, Homo sapiens
WGDEGFDI

SEQ ID NO: 131 - protein, 11 aa, Homo sapiens
RASQGISNWLA

SEQ ID NO: 132 - protein, 7 aa, Homo sapiens
GASSLQS

SEQ ID NO: 133 - protein, 9 aa, Homo sapiens
QQYSSFPTT

SEQ ID NO: 134 - protein, 7 aa, Homo sapiens
GFTFSSY

SEQ ID NO: 135 - protein, 6 aa, Homo sapiens
NSQGKS

SEQ ID NO: 136 - protein, 8 aa, Homo sapiens
WGDEGFDI

SEQ ID NO: 137 - protein, 7 aa, Homo sapiens
SQGISNW

SEQ ID NO: 138 - protein, 3 aa, Homo sapiens
GAS

SEQ ID NO: 139 - protein, 6 aa, Homo sapiens
YSSFPT

SEQ ID NO: 140 - protein, 107 aa, Homo sapiens; identical to SEQ ID NO: 104

SEQ ID NO: 141 - protein, 117 aa, Homo sapiens
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSS

SEQ ID NO: 142 - DNA, 321 bp, Homo sapiens; identical to SEQ ID NO: 106

SEQ ID NO: 143 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgccatcaacagccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 144 - protein, 214 aa, Homo sapiens; identical to SEQ ID NO: 108

SEQ ID NO: 145 - protein, 447 aa, Homo sapiens
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 5 aa, Homo sapiens
SYAMS

SEQ ID NO: 147 - protein, 17 aa, Homo sapiens
AISSQGKSTYYADSVKG

SEQ ID NO: 148 - protein, 8 aa, Homo sapiens
WGDEGFDI

SEQ ID NO: 149 - protein, 11 aa, Homo sapiens
RASQGISNWLA

SEQ ID NO: 150 - protein, 7 aa, Homo sapiens
GASSLQS

SEQ ID NO: 151 - protein, 9 aa, Homo sapiens
QQYSSFPTT

SEQ ID NO: 152 - protein, 7 aa, Homo sapiens
GFTFSSY

SEQ ID NO: 153 - protein, 6 aa, Homo sapiens
SSQGKS

SEQ ID NO: 154 - protein, 8 aa, Homo sapiens
WGDEGFDI

SEQ ID NO: 155 - protein, 7 aa, Homo sapiens
SQGISNW

SEQ ID NO: 156 - protein, 3 aa, Homo sapiens
GAS

SEQ ID NO: 157 - protein, 6 aa, Homo sapiens
YSSFPT

SEQ ID NO: 158 - protein, 107 aa, Homo sapiens; identical to SEQ ID NO: 104

SEQ ID NO: 159 - protein, 117 aa, Homo sapiens
EVQLLESGGGLVQPGGSLRLSCAASGFTFSSYAMSWVRQAPGKGLEWVSAISSQGKSTYYADSVKGRFTISRDNSKNTLYLQMNSLRAEDTAVYYCARWGDEGFDIWGQGTLVTVSS

SEQ ID NO: 160 - DNA, 321 bp, Homo sapiens; identical to SEQ ID NO: 106

SEQ ID NO: 161 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgccatcagcagccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 162 - protein, 214 aa, Homo sapiens; identical to SEQ ID NO: 108

SEQ ID NO: 163 - protein, 447 aa, Homo sapiens
EVQLLESGGGLVQPGGSLRLSCAASGFTFSSYAMSWVRQAPGKGLEWVSAISSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 5 aa, Homo sapiens
SYAMS

SEQ ID NO: 165 - protein, 17 aa, Homo sapiens
AIGSQGKSTYYADSVKG

SEQ ID NO: 166 - protein, 8 aa, Homo sapiens
WGDEGFDI

SEQ ID NO: 167 - protein, 11 aa, Homo sapiens
RASQGISNWLA

SEQ ID NO: 168 - protein, 7 aa, Homo sapiens
GASSLQS

SEQ ID NO: 169 - protein, 9 aa, Homo sapiens
QQYSSFPTT

SEQ ID NO: 170 - protein, 7 aa, Homo sapiens
GFTFSSY

SEQ ID NO: 171 - protein, 6 aa, Homo sapiens
GSQGKS

SEQ ID NO: 172 - protein, 8 aa, Homo sapiens
WGDEGFDI

SEQ ID NO: 173 - protein, 7 aa, Homo sapiens
SQGISNW

SEQ ID NO: 174 - protein, 3 aa, Homo sapiens
GAS

SEQ ID NO: 175 - protein, 6 aa, Homo sapiens
YSSFPT

SEQ ID NO: 176 - protein, 107 aa, Homo sapiens; identical to SEQ ID NO: 104

SEQ ID NO: 177 - protein, 117 aa, Homo sapiens
EVQLLESGGGLVQPGGSLRLSCAASGFTFSSYAMSWVRQAPGKGLEWVSAIGSQGKSTYYADSVKGRFTISRDNSKNTLYLQMNSLRAEDTAVYYCARWGDEGFDIWGQGTLVTVSS

SEQ ID NO: 178 - DNA, 321 bp, Homo sapiens; identical to SEQ ID NO: 106

SEQ ID NO: 179 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgccatcggcagccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 180 - protein, 214 aa, Homo sapiens; identical to SEQ ID NO: 108

SEQ ID NO: 181 - protein, 447 aa, Homo sapiens
EVQLLESGGGLVQPGGSLRLSCAASGFTFSSYAMSWVRQAPGKGLEWVSAIGSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 5 aa, Homo sapiens
SYAMS

SEQ ID NO: 183 - protein, 17 aa, Homo sapiens
AISNQGKSTYYADSVKG

SEQ ID NO: 184 - protein, 8 aa, Homo sapiens
WGDEGFDI

SEQ ID NO: 185 - protein, 11 aa, Homo sapiens
RASQGISNWLA

SEQ ID NO: 186 - protein, 7 aa, Homo sapiens
GASSLQS

SEQ ID NO: 187 - protein, 9 aa, Homo sapiens
QQYSSFPTT

SEQ ID NO: 188 - protein, 7 aa, Homo sapiens
GFTFSSY

SEQ ID NO: 189 - protein, 6 aa, Homo sapiens
SNQGKS

SEQ ID NO: 190 - protein, 8 aa, Homo sapiens
WGDEGFDI

SEQ ID NO: 191 - protein, 7 aa, Homo sapiens
SQGISNW

SEQ ID NO: 192 - protein, 3 aa, Homo sapiens
GAS

SEQ ID NO: 193 - protein, 6 aa, Homo sapiens
YSSFPT

SEQ ID NO: 194 - protein, 107 aa, Homo sapiens; identical to SEQ ID NO: 104

SEQ ID NO: 195 - protein, 117 aa, Homo sapiens
EVQLLESGGGLVQPGGSLRLSCAASGFTFSSYAMSWVRQAPGKGLEWVSAISNQGKSTYYADSVKGRFTISRDNSKNTLYLQMNSLRAEDTAVYYCARWGDEGFDIWGQGTLVTVSS

SEQ ID NO: 196 - DNA, 321 bp, Homo sapiens; identical to SEQ ID NO: 106

SEQ ID NO: 197 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgccatcagcaaccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 198 - protein, 214 aa, Homo sapiens; identical to SEQ ID NO: 108

SEQ ID NO: 199 - protein, 447 aa, Homo sapiens
EVQLLESGGGLVQPGGSLRLSCAASGFTFSSYAMSWVRQAPGKGLEWVSAISNQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 5 aa, Homo sapiens
SYAMS

SEQ ID NO: 201 - protein, 17 aa, Homo sapiens
VISSQGKSTYYADSVKG

SEQ ID NO: 202 - protein, 8 aa, Homo sapiens
WGDEGFDI

SEQ ID NO: 203 - protein, 11 aa, Homo sapiens
RASQGISNWLA

SEQ ID NO: 204 - protein, 7 aa, Homo sapiens
GASSLQS

SEQ ID NO: 205 - protein, 9 aa, Homo sapiens
QQYSSFPTT

SEQ ID NO: 206 - protein, 7 aa, Homo sapiens
GFTFSSY

SEQ ID NO: 207 - protein, 6 aa, Homo sapiens
SSQGKS

SEQ ID NO: 208 - protein, 8 aa, Homo sapiens
WGDEGFDI

SEQ ID NO: 209 - protein, 7 aa, Homo sapiens
SQGISNW

SEQ ID NO: 210 - protein, 3 aa, Homo sapiens
GAS

SEQ ID NO: 211 - protein, 6 aa, Homo sapiens
YSSFPT

SEQ ID NO: 212 - protein, 107 aa, Homo sapiens; identical to SEQ ID NO: 104

SEQ ID NO: 213 - protein, 117 aa, Homo sapiens
EVQLLESGGGLVQPGGSLRLSCAASGFTFSSYAMSWVRQAPGKGLEWVSVISSQGKSTYYADSVKGRFTISRDNSKNTLYLQMNSLRAEDTAVYYCARWGDEGFDIWGQGTLVTVSS

SEQ ID NO: 214 - DNA, 321 bp, Homo sapiens; identical to SEQ ID NO: 106

SEQ ID NO: 215 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtcatcagcagccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 216 - protein, 214 aa, Homo sapiens; identical to SEQ ID NO: 108

SEQ ID NO: 217 - protein, 447 aa, Homo sapiens
EVQLLESGGGLVQPGGSLRLSCAASGFTFSSYAMSWVRQAPGKGLEWVSVISSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 5 aa, Homo sapiens
SYAMS

SEQ ID NO: 219 - protein, 17 aa, Homo sapiens
VIGSQGKSTYYADSVKG

SEQ ID NO: 220 - protein, 8 aa, Homo sapiens
WGDEGFDI

SEQ ID NO: 221 - protein, 11 aa, Homo sapiens
RASQGISNWLA

SEQ ID NO: 222 - protein, 7 aa, Homo sapiens
GASSLQS

SEQ ID NO: 223 - protein, 9 aa, Homo sapiens
QQYSSFPTT

SEQ ID NO: 224 - protein, 7 aa, Homo sapiens
GFTFSSY

SEQ ID NO: 225 - protein, 6 aa, Homo sapiens
GSQGKS

SEQ ID NO: 226 - protein, 8 aa, Homo sapiens
WGDEGFDI

SEQ ID NO: 227 - protein, 7 aa, Homo sapiens
SQGISNW

SEQ ID NO: 228 - protein, 3 aa, Homo sapiens
GAS

SEQ ID NO: 229 - protein, 6 aa, Homo sapiens
YSSFPT

SEQ ID NO: 230 - protein, 107 aa, Homo sapiens; identical to SEQ ID NO: 104

SEQ ID NO: 231 - protein, 117 aa, Homo sapiens
EVQLLESGGGLVQPGGSLRLSCAASGFTFSSYAMSWVRQAPGKGLEWVSVIGSQGKSTYYADSVKGRFTISRDNSKNTLYLQMNSLRAEDTAVYYCARWGDEGFDIWGQGTLVTVSS

SEQ ID NO: 232 - DNA, 321 bp, Homo sapiens; identical to SEQ ID NO: 106

SEQ ID NO: 233 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtcatcggcagccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 234 - protein, 214 aa, Homo sapiens; identical to SEQ ID NO: 108

SEQ ID NO: 235 - protein, 447 aa, Homo sapiens
EVQLLESGGGLVQPGGSLRLSCAASGFTFSSYAMSWVRQAPGKGLEWVSVIGSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 5 aa, Homo sapiens
SYAMS

SEQ ID NO: 237 - protein, 17 aa, Homo sapiens
AINAQGKSTYYADSVKG

SEQ ID NO: 238 - protein, 8 aa, Homo sapiens
WGDEGFDI

SEQ ID NO: 239 - protein, 11 aa, Homo sapiens
RASQGISNWLA

SEQ ID NO: 240 - protein, 7 aa, Homo sapiens
GASSLQS

SEQ ID NO: 241 - protein, 9 aa, Homo sapiens
QQYSSFPTT

SEQ ID NO: 242 - protein, 7 aa, Homo sapiens
GFTFSSY

SEQ ID NO: 243 - protein, 6 aa, Homo sapiens
NAQGKS

SEQ ID NO: 244 - protein, 8 aa, Homo sapiens
WGDEGFDI

SEQ ID NO: 245 - protein, 7 aa, Homo sapiens
SQGISNW

SEQ ID NO: 246 - protein, 3 aa, Homo sapiens
GAS

SEQ ID NO: 247 - protein, 6 aa, Homo sapiens
YSSFPT

SEQ ID NO: 248 - protein, 107 aa, Homo sapiens; identical to SEQ ID NO: 104

SEQ ID NO: 249 - protein, 117 aa, Homo sapiens
EVQLLESGGGLVQPGGSLRLSCAASGFTFSSYAMSWVRQAPGKGLEWVSAINAQGKSTYYADSVKGRFTISRDNSKNTLYLQMNSLRAEDTAVYYCARWGDEGFDIWGQGTLVTVSS

SEQ ID NO: 250 - DNA, 321 bp, Homo sapiens; identical to SEQ ID NO: 106

SEQ ID NO: 251 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgccatcaacgcccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 252 - protein, 214 aa, Homo sapiens; identical to SEQ ID NO: 108

SEQ ID NO: 253 - protein, 447 aa, Homo sapiens
EVQLLESGGGLVQPGGSLRLSCAASGFTFSSYAMSWVRQAPGKGLEWVSAINAQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 5 aa, Homo sapiens
SYAMS

SEQ ID NO: 255 - protein, 17 aa, Homo sapiens
AINTQGKSTYYADSVKG

SEQ ID NO: 256 - protein, 8 aa, Homo sapiens
WGDEGFDI

SEQ ID NO: 257 - protein, 11 aa, Homo sapiens
RASQGISNWLA

SEQ ID NO: 258 - protein, 7 aa, Homo sapiens
GASSLQS

SEQ ID NO: 259 - protein, 9 aa, Homo sapiens
QQYSSFPTT

SEQ ID NO: 260 - protein, 7 aa, Homo sapiens
GFTFSSY

SEQ ID NO: 261 - protein, 6 aa, Homo sapiens
NTQGKS

SEQ ID NO: 262 - protein, 8 aa, Homo sapiens
WGDEGFDI

SEQ ID NO: 263 - protein, 7 aa, Homo sapiens
SQGISNW

SEQ ID NO: 264 - protein, 3 aa, Homo sapiens
GAS

SEQ ID NO: 265 - protein, 6 aa, Homo sapiens
YSSFPT

SEQ ID NO: 266 - protein, 107 aa, Homo sapiens; identical to SEQ ID NO: 104

SEQ ID NO: 267 - protein, 117 aa, Homo sapiens
EVQLLESGGGLVQPGGSLRLSCAASGFTFSSYAMSWVRQAPGKGLEWVSAINTQGKSTYYADSVKGRFTISRDNSKNTLYLQMNSLRAEDTAVYYCARWGDEGFDIWGQGTLVTVSS

SEQ ID NO: 268 - DNA, 321 bp, Homo sapiens; identical to SEQ ID NO: 106

SEQ ID NO: 269 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgccatcaacacccagggcaagagcacctactacgccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 270 - protein, 214 aa, Homo sapiens; identical to SEQ ID NO: 108

SEQ ID NO: 271 - protein, 447 aa, Homo sapiens
EVQLLESGGGLVQPGGSLRLSCAASGFTFSSYAMSWVRQAPGKGLEWVSAINTQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 5 aa, Homo sapiens
SYAMS

SEQ ID NO: 273 - protein, 17 aa, Homo sapiens
VTGAVGSSTYYPDSVKG

SEQ ID NO: 274 - protein, 8 aa, Homo sapiens
WGDEGFDI

SEQ ID NO: 275 - protein, 11 aa, Homo sapiens
RASQGISNWLA

SEQ ID NO: 276 - protein, 7 aa, Homo sapiens
GASSLQS

SEQ ID NO: 277 - protein, 9 aa, Homo sapiens
QQYSSFPTT

SEQ ID NO: 278 - protein, 7 aa, Homo sapiens
GFTFSSY

SEQ ID NO: 279 - protein, 6 aa, Homo sapiens
GAVGSS

SEQ ID NO: 280 - protein, 8 aa, Homo sapiens
WGDEGFDI

SEQ ID NO: 281 - protein, 7 aa, Homo sapiens
SQGISNW

SEQ ID NO: 282 - protein, 3 aa, Homo sapiens
GAS

SEQ ID NO: 283 - protein, 6 aa, Homo sapiens
YSSFPT

SEQ ID NO: 284 - protein, 107 aa, Homo sapiens; identical to SEQ ID NO: 104

SEQ ID NO: 285 - protein, 117 aa, Homo sapiens
EVQLLESGGGLVQPGGSLRLSCAASGFTFSSYAMSWVRQAPGKGLEWVSVTGAVGSSTYYPDSVKGRFTISRDNSKNTLYLQMNSLRAEDTAVYYCARWGDEGFDIWGQGTLVTVSS

SEQ ID NO: 286 - DNA, 321 bp, Homo sapiens; identical to SEQ ID NO: 106

SEQ ID NO: 287 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtgacaggcgccgtgggcagcagcacctactaccccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 288 - protein, 214 aa, Homo sapiens; identical to SEQ ID NO: 108

SEQ ID NO: 289 - protein, 447 aa, Homo sapiens
EVQLLESGGGLVQPGGSLRLSCAASGFTFSSYAMSWVRQAPGKGLEWVSVTGAVGSSTYYPDSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - protein, 5 aa, Homo sapiens
SYAMS

SEQ ID NO: 291 - protein, 17 aa, Homo sapiens
VTGAVGGSTYYPDSVKG

SEQ ID NO: 292 - protein, 8 aa, Homo sapiens
WGDEGFDI

SEQ ID NO: 293 - protein, 11 aa, Homo sapiens
RASQGISNWLA

SEQ ID NO: 294 - protein, 7 aa, Homo sapiens
GASSLQS

SEQ ID NO: 295 - protein, 9 aa, Homo sapiens
QQYSSFPTT

SEQ ID NO: 296 - protein, 7 aa, Homo sapiens
GFTFSSY

SEQ ID NO: 297 - protein, 6 aa, Homo sapiens
GAVGGS

SEQ ID NO: 298 - protein, 8 aa, Homo sapiens
WGDEGFDI

SEQ ID NO: 299 - protein, 7 aa, Homo sapiens
SQGISNW

SEQ ID NO: 300 - protein, 3 aa, Homo sapiens
GAS

SEQ ID NO: 301 - protein, 6 aa, Homo sapiens
YSSFPT

SEQ ID NO: 302 - protein, 107 aa, Homo sapiens; identical to SEQ ID NO: 104

SEQ ID NO: 303 - protein, 117 aa, Homo sapiens
EVQLLESGGGLVQPGGSLRLSCAASGFTFSSYAMSWVRQAPGKGLEWVSVTGAVGGSTYYPDSVKGRFTISRDNSKNTLYLQMNSLRAEDTAVYYCARWGDEGFDIWGQGTLVTVSS

SEQ ID NO: 304 - DNA, 321 bp, Homo sapiens; identical to SEQ ID NO: 106

SEQ ID NO: 305 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtgacaggcgccgtgggcggaagcacctactaccccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 306 - protein, 214 aa, Homo sapiens; identical to SEQ ID NO: 108

SEQ ID NO: 307 - protein, 447 aa, Homo sapiens
EVQLLESGGGLVQPGGSLRLSCAASGFTFSSYAMSWVRQAPGKGLEWVSVTGAVGGSTYYPDSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 5 aa, Homo sapiens
SYAMS

SEQ ID NO: 309 - protein, 17 aa, Homo sapiens
VTGAVGKSTYYPDSVKG

SEQ ID NO: 310 - protein, 8 aa, Homo sapiens
WGDEGFDI

SEQ ID NO: 311 - protein, 11 aa, Homo sapiens
RASQGISNWLA

SEQ ID NO: 312 - protein, 7 aa, Homo sapiens
GASSLQS

SEQ ID NO: 313 - protein, 9 aa, Homo sapiens
QQYSSFPTT

SEQ ID NO: 314 - protein, 7 aa, Homo sapiens
GFTFSSY

SEQ ID NO: 315 - protein, 6 aa, Homo sapiens
GAVGKS

SEQ ID NO: 316 - protein, 8 aa, Homo sapiens
WGDEGFDI

SEQ ID NO: 317 - protein, 7 aa, Homo sapiens
SQGISNW

SEQ ID NO: 318 - protein, 3 aa, Homo sapiens
GAS

SEQ ID NO: 319 - protein, 6 aa, Homo sapiens
YSSFPT

SEQ ID NO: 320 - protein, 107 aa, Homo sapiens; identical to SEQ ID NO: 104

SEQ ID NO: 321 - protein, 117 aa, Homo sapiens
EVQLLESGGGLVQPGGSLRLSCAASGFTFSSYAMSWVRQAPGKGLEWVSVTGAVGKSTYYPDSVKGRFTISRDNSKNTLYLQMNSLRAEDTAVYYCARWGDEGFDIWGQGTLVTVSS

SEQ ID NO: 322 - DNA, 321 bp, Homo sapiens; identical to SEQ ID NO: 106

SEQ ID NO: 323 - DNA, 351 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtgacaggcgccgtgggcaaaagcacctactaccccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 324 - protein, 214 aa, Homo sapiens; identical to SEQ ID NO: 108

SEQ ID NO: 325 - protein, 447 aa, Homo sapiens
EVQLLESGGGLVQPGGSLRLSCAASGFTFSSYAMSWVRQAPGKGLEWVSVTGAVGKSTYYPDSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 5 aa, Homo sapiens
SYAMS

SEQ ID NO: 327 - protein, 16 aa, Homo sapiens
VTGAVGRTYYPDSVKG

SEQ ID NO: 328 - protein, 8 aa, Homo sapiens
WGDEGFDI

SEQ ID NO: 329 - protein, 11 aa, Homo sapiens
RASQGISNWLA

SEQ ID NO: 330 - protein, 7 aa, Homo sapiens
GASSLQS

SEQ ID NO: 331 - protein, 9 aa, Homo sapiens
QQYSSFPTT

SEQ ID NO: 332 - protein, 7 aa, Homo sapiens
GFTFSSY

SEQ ID NO: 333 - protein, 6 aa, Homo sapiens
GAVGRT

SEQ ID NO: 334 - protein, 8 aa, Homo sapiens
WGDEGFDI

SEQ ID NO: 335 - protein, 7 aa, Homo sapiens
SQGISNW

SEQ ID NO: 336 - protein, 3 aa, Homo sapiens
GAS

SEQ ID NO: 337 - protein, 6 aa, Homo sapiens
YSSFPT

SEQ ID NO: 338 - protein, 107 aa, Homo sapiens; identical to SEQ ID NO: 104

SEQ ID NO: 339 - protein, 116 aa, Homo sapiens
EVQLLESGGGLVQPGGSLRLSCAASGFTFSSYAMSWVRQAPGKGLEWVSVTGAVGRTYYPDSVKGRFTISRDNSKNTLYLQMNSLRAEDTAVYYCARWGDEGFDIWGQGTLVTVSS

SEQ ID NO: 340 - DNA, 321 bp, Homo sapiens; identical to SEQ ID NO: 106

SEQ ID NO: 341 - DNA, 348 bp, Homo sapiens
gaggtgcaattgctggaaagcggcggaggcctggtgcagcctggcggcagcctgagactgtcttgcgccgccagcggcttcaccttcagcagctacgccatgagctgggtccgccaggcccctggcaagggactggaatgggtgtccgtgacaggcgccgtgggcagaacctactaccccgacagcgtgaagggccggttcaccatcagccgggacaacagcaagaacaccctgtacctgcagatgaacagcctgcgggccgaggacaccgccgtgtactactgtgccagatggggcgacgagggcttcgacatctggggccagggcaccctggtcaccgtcagctca

SEQ ID NO: 342 - protein, 214 aa, Homo sapiens; identical to SEQ ID NO: 108

SEQ ID NO: 343 - protein, 446 aa, Homo sapiens
EVQLLESGGGLVQPGGSLRLSCAASGFTFSSYAMSWVRQAPGKGLEWVSVTGAVGRTYYPDSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 5 aa, Homo sapiens
SYAMS

SEQ ID NO: 345 - protein, 17 aa, Homo sapiens
VINGLGYTTFYADSVKG

SEQ ID NO: 346 - protein, 8 aa, Homo sapiens
WGDEGFDI

SEQ ID NO: 347 - protein, 11 aa, Homo sapiens
RASQGISNWLA

SEQ ID NO: 348 - protein, 7 aa, Homo sapiens
GASSLQS

SEQ ID NO: 349 - protein, 9 aa, Homo sapiens
QQYSSFPTT

SEQ ID NO: 350 - protein, 7 aa, Homo sapiens
GFTFSSY

SEQ ID NO: 351 - protein, 6 aa, Homo sapiens
NGLGYT

SEQ ID NO: 352 - protein, 8 aa, Homo sapiens
WGDEGFDI

SEQ ID NO: 353 - protein, 7 aa, Homo sapiens
SQGISNW

SEQ ID NO: 354 - protein, 3 aa, Homo sapiens
GAS

SEQ ID NO: 355 - protein, 6 aa, Homo sapiens
YSSFPT

SEQ ID NO: 356 - protein, 107 aa, Homo sapiens; identical to SEQ ID NO: 14

SEQ ID NO: 357 - protein, 117 aa, Homo sapiens
QVQLVESGGGLVQPGGSLRLSCAASGFTFSSYAMSWVRQAPGKGLEWVSVINGLGYTTFYADSVKGRFTISRDNSKNTLYLQMNSLRAEDTAVYYCARWGDEGFDIWGQGTLVTVSS

SEQ ID NO: 358 - DNA, 321 bp, Homo sapiens; identical to SEQ ID NO: 16

SEQ ID NO: 359 - DNA, 351 bp, Homo sapiens
caggtgcaattggtggaaagcggcggcggcctggtgcaaccgggcggcagcctgcgtctgagctgcgcggcctccggatttacctttagcagctatgcgatgagctgggtgcgccaagcccctgggaagggtctcgagtgggtgagcgttattaatggtcttggttatactactttttatgctgattctgttaagggtcgttttaccatttcacgtgataattcgaaaaacaccctgtatctgcaaatgaacagcctgcgtgcggaagatacggccgtgtattattgcgcgcgttggggtgatgagggttttgatatttggggccaaggcaccctggtgacggttagctca

SEQ ID NO: 360 - protein, 214 aa, Homo sapiens; identical to SEQ ID NO: 18

SEQ ID NO: 361 - protein, 447 aa, Homo sapiens
QVQLVESGGGLVQPGGSLRLSCAASGFTFSSYAMSWVRQAPGKGLEWVSVINGLGYTTFYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 5 aa, Homo sapiens
SYAMS

SEQ ID NO: 363 - protein, 16 aa, Homo sapiens
GTGPYGGTYYPDSVKG

SEQ ID NO: 364 - protein, 8 aa, Homo sapiens
WGDEGFDI

SEQ ID NO: 365 - protein, 11 aa, Homo sapiens
RASQGISNWLA

SEQ ID NO: 366 - protein, 7 aa, Homo sapiens
GASSLQS

SEQ ID NO: 367 - protein, 9 aa, Homo sapiens
QQYSSFPTT

SEQ ID NO: 368 - protein, 7 aa, Homo sapiens
GFTFSSY

SEQ ID NO: 369 - protein, 5 aa, Homo sapiens
GPYGG

SEQ ID NO: 370 - protein, 8 aa, Homo sapiens
WGDEGFDI

SEQ ID NO: 371 - protein, 7 aa, Homo sapiens
SQGISNW

SEQ ID NO: 372 - protein, 3 aa, Homo sapiens
GAS

SEQ ID NO: 373 - protein, 6 aa, Homo sapiens
YSSFPT

SEQ ID NO: 374 - protein, 107 aa, Homo sapiens; identical to SEQ ID NO: 14

SEQ ID NO: 375 - protein, 116 aa, Homo sapiens
QVQLVESGGGLVQPGGSLRLSCAASGFTFSSYAMSWVRQAPGKGLEWVSGTGPYGGTYYPDSVKGRFTISRDNSKNTLYLQMNSLRAEDTAVYYCARWGDEGFDIWGQGTLVTVSS

SEQ ID NO: 376 - DNA, 321 bp, Homo sapiens; identical to SEQ ID NO: 16

SEQ ID NO: 377 - DNA, 348 bp, Homo sapiens
caggtgcaattggtggaaagcggcggcggcctggtgcaaccgggcggcagcctgcgtctgagctgcgcggcctccggatttacctttagcagctatgcgatgagctgggtgcgccaagcccctgggaagggtctcgagtgggtgagcggtactggtccttatggtggtacttattatcctgattctgttaagggtcgttttaccatttcacgtgataattcgaaaaacaccctgtatctgcaaatgaacagcctgcgtgcggaagatacggccgtgtattattgcgcgcgttggggtgatgagggttttgatatttggggccaaggcaccctggtgacggttagctca

SEQ ID NO: 378 - protein, 214 aa, Homo sapiens; identical to SEQ ID NO: 18

SEQ ID NO: 379 - protein, 446 aa, Homo sapiens
QVQLVESGGGLVQPGGSLRLSCAASGFTFSSYAMSWVRQAPGKGLEWVSGTGPYGGTYYPDSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2017/0252335 A1
(43) Pub. Date: Sep. 7, 2017

(54) Combination of Ceritinib with an EGFR Inhibitor

(71) Applicants: Serafino Pantano, Bartenheim (FR); Fang Li, Arlington, MA (US); Nanxin Li, San Diego, CA (US); Anthony Boral, Cambridge, MA (US)

(72) Inventors: Serafino Pantano, Bartenheim (FR); Fang Li, Arlington, MA (US); Nanxin Li, San Diego, CA (US); Anthony Boral, Cambridge, MA (US)

(73) Assignee: Novartis AG, Basel (CH)

(21) Appl. No.: 15/519,593

(22) PCT Filed: Oct. 15, 2015
(86) PCT No.: PCT/IB2015/057940

§ 371 Date: Apr. 17, 2017

Related U.S. Application Data
(60) Provisional application No. 62/065,451, filed on Oct. 17, 2014.
(60) Provisional application No. 62/068,175, filed on Oct. 24, 2014.
(60) Provisional application No. 62/068,945, filed on Oct. 27, 2014.
(60) Provisional application No. 62/118,710, filed on Feb. 20, 2015.

(51) Int. Cl. A61K 31/4706; A61K 31/4709; A61K 31/506; C07K 16/32; A61K 31/519; A61K 31/5377; A61K 31/55; A61K 39/395; A61K 31/517

(52) U.S. Cl. CPC A61K 31/4706; A61K 39/39558; A61K 31/4709; A61K 31/506; A61K 31/517; A61K 31/519; A61K 31/5377; A61K 31/55; C07K 16/32; A61K 45/06

(57) ABSTRACT

The present disclosure relates to a pharmaceutical composition comprising two Tyrosine Kinase Inhibitors (TKIs), namely Ceritinib and an EGFR Inhibitor. The present combination can be administered independently or separately, in a quantity which is jointly therapeutically effective for the treatment of a TKI mediated disease, such as cancer. The disclosure also provides the use of such a combination for the manufacture of a medicament; the use of such a combination as a medicine; a kit of part comprising such a combination; and a method of treatment of such a combination.

### FIELD OF THE DISCLOSURE

The present disclosure relates to a pharmaceutical composition comprising two Tyrosine Kinase Inhibitors (TKIs). The present combination is administered independently or separately, in a quantity which is jointly therapeutically effective for the treatment of a TKI mediated disease. The disclosure further relates to a use of such combination for the manufacture of a medicament; the use of such combination as a medicine; a kit of parts comprising such a combination; and a method of treatment involving the combination.

### BACKGROUND OF THE DISCLOSURE

Targeted therapies, such as Tyrosine Kinase Inhibitors (TKIs), are widely used to treat several types of cancers and offer an alternative to standard platinum-based chemotherapy.

ALK is a member of the insulin receptor superfamily of receptor tyrosine kinases. Chromosomal rearrangements involving anaplastic lymphoma kinase (ALK) has been detected in a variety of human malignancies, leading to disturbances in the regulation pathway of the cells. Inhibition or suppression of the ALK pathways using an ALK tyrosine kinase inhibitor engenders the cell growth arrest and apoptosis of malignant cells. Targeted therapies involving ALK tyrosine kinase inhibitors have been developed.

Ceritinib (LDK378) is an Anaplastic Lymphoma Kinase (ALK) inhibitor. Its chemical formula is 5-chloro-N²-(2-isopropoxy-5-methyl-4-(piperidin-4-yl)phenyl)-N⁴-[2-(propane-2-sulfonyl)-phenyl]-pyrimidine-2,4-diamine. A process for preparing Ceritinib was disclosed in WO2008/073687. The compound has been approved by the US FDA as Zykadia® for the treatment of patients with Anaplastic Lymphoma Kinase (ALK)-positive metastatic non-small cell lung cancer (NSCLC), who have progressed on or are intolerant to crizotinib.

The Epidermal Growth Factor Receptor is a receptor for ligands of the epidermal growth factor family with several family members. Several types of cancers are known to be dependent on EGFR over-activity or over-expression.

### BRIEF DESCRIPTION OF THE DISCLOSURE

It was observed that EGFR signaling can bypass the ALK inhibition. This can happen in EGFR mutant cancer types as well as cancers with no EGFR mutation. Based on clinical experience with ALK inhibitors it was observed that patients with ALK mutations developed resistance to the targeted therapies ALK tyrosine kinase inhibitors, 1 to 2 years following the start of the treatment. Surprisingly, it was observed that the acquired resistance was not only due to the development of secondary ALK mutations but also the emergence of other oncogenic drivers such as EGFR.

EGFR inhibitor alone is not sufficient when the cells acquired resistance to the treatment with an ALK inhibitor. The combination of an ALK inhibitor and EGFR inhibitor is required. Therefore, the present disclosure deals with a combination of ceritinib and an EGFR inhibitor that can provide an advantageous effect from the start of the treatment. It became clear that the combination would be valuable in the treatment of an ALK-naïve patient, for example where the patient has previously not been treated with an ALK inhibitor. In addition, the combination overcomes possible acquired resistance in ALK-positive cancers. The combination would thus be useful in post ALK inhibitor setting, such as post crizotinib or post ceritinib setting.

The first aspect of the present disclosure is a pharmaceutical combination comprising (i) ceritinib, or a pharmaceutically acceptable salt thereof, and (ii) an EGFR inhibitor, or a pharmaceutically acceptable salt thereof.

Another aspect of this disclosure is a use of ceritinib in combination with an EGFR inhibitor for the manufacture of a medicament for an ALK and/or EGFR mediated disease.

A further aspect of the disclosure provides a pharmaceutical composition comprising effective amounts of ceritinib or a pharmaceutically acceptable salt thereof, and an EGFR inhibitor or a pharmaceutically acceptable salt thereof, for simultaneous or separate administration for the treatment of cancer.

A yet another aspect of the disclosure is ceritinib for use as a medicine, wherein ceritinib, or a pharmaceutically acceptable salt thereof, is to be administered in combination with an EGFR inhibitor, or a pharmaceutically acceptable salt thereof.

### BRIEF DESCRIPTION OF FIGURES

FIG. 1 depicts a scatter plot obtained by the Cell Titer Glo assay on H2228 cell line showing which ligands could reverse the growth inhibitory effect of ceritinib.

FIG. 2 depicts cell proliferation of MGH049 and MGH051 cells treated with the indicated doses of ceritinib for 6 days.

FIG. 3 depicts effects on MGH049 cell growth by the EGFR inhibitors alone (top line of the graphs) or EGFR inhibitors in combination with 0.5 μM ceritinib (bottom line on the graphs).

FIG. 4 depicts effects on MGH051 cell growth by the EGFR inhibitors alone (top line of the graphs) or EGFR inhibitors in combination with 0.5 μM ceritinib (bottom line on the graphs).

FIG. 5. Inhibition of ALK induces activation of HER3 in ALK positive NSCLC cell lines. Cells were harvested after short-term and long-term ceritinib (LDK378) treatment, and whole cell lysates were analyzed by Western blotting. Long-term ALK inhibition led to up-regulation of HER3 phosphorylation.

FIG. 6. Addition of HER3 and EGFR inhibitors represses the rebound in AKT phosphorylation. MGH051 cells were treated with ceritinib (LDK378) for 4 hours or 8 days. Antibody A, erlotinib and afatinib were added for the last day of the long-term LDK378 treatment.

FIG. 7. Erlotinib enhances the anti-proliferation activity of ceritinib (LDK378) in ALK positive NSCLC cells. Cells were exposed to ceritinib (0.5 μM), erlotinib (1 μM), or the combination for 7 days. For each cell line, all dishes were fixed, stained and photographed at the same time.

FIGS. 8A and 8B. Efficacy of ceritinib (LDK378) alone and in combination with cetuximab and/or Antibody A (Ab A; MOR10703 from table 1) in H2228 NSCLC xenograft in female SCID-beige mice.

### SPECIFIC DESCRIPTION OF THE DISCLOSURE

It was surprisingly found that EGFR signaling can bypass the ALK inhibition. Results from in vitro and in vivo studies in ALK-positive NSCLC cell lines showed that inhibition of EGFR and ALK worked synergic in settings where an ALK inhibitor has not been used and where the model already initially showed resistance to an ALK inhibitor. Surprisingly, those studies also lead to the discovery of a synergistic anti-cancer drug combinations able to overcome possible resistance pathway induced while treating the ALK mutated cell with ceritinib. It was found that the resistance could be overcome by combining ceritinib with an EGFR inhibitor. And it was also found that acquired resistance to ceritinib could be overcome by combining ceritinib with an EGFR inhibitor to prevent EGFR pathway from bypassing the ALK signaling in ALK positive NSCLC. The synergistic combination of ceritinib abd an EGFR inhibitor according to the disclosure can be administered independently at the same time or separately within time intervals. Therefore, the present disclosure provides a pharmaceutical combination (e.g. a combination product) comprising (i) ceritinib, or a pharmaceutically acceptable salt thereof, and (ii) an EGFR inhibitor, or a pharmaceutically acceptable salt thereof.

Pharmaceutically acceptable salts can be formed, for example, as acid addition salts, preferably with organic or inorganic acids. Suitable inorganic acids are, for example, halogen acids, such as hydrochloric acid. Suitable organic acids are, e.g., carboxylic acids or sulfonic acids, such as fumaric acid or methanesulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. The salts of compounds of formula (I) are preferably pharmaceutically acceptable salts; suitable counter-ions forming pharmaceutically acceptable salts are known in the field.

The present disclosure, according to a first embodiment mentioned above, relates to a pharmaceutical combination, especially a pharmaceutical combination product, comprising the mentioned combination partners.

EGFR inhibitor can be any compound that targets, decreases or inhibits the activity of the epidermal growth factor family of receptor tyrosine kinases (EGFR (ErbB1), ErbB2, ErbB3, ErbB4 as homo- or heterodimers) and their mutants. EGFR (ErbB1, HER1), ErbB2 (HER2), ErbB3 (HER3) and ErbB4 (HER4) are structurally related single chain transmembrane glycoprotein receptors consisting of an extracellular ligand-binding ectodomain, a transmembrane domain, a short juxtamembrane section, a tyrosine kinase domain and a tyrosine-containing C-terminal tail. Compounds which target, decrease or inhibit the activity of the epidermal growth factor receptor family are especially compounds, proteins or antibodies which inhibit members of the EGF receptor tyrosine kinase family, e.g. EGF receptor (ErbB1), ErbB2, ErbB3 and ErbB4 or bind to EGF or EGF related ligands. In a particular embodiment, EGFR inhibitor targets, decreases or inhibits the activity of EGFR, aka ErbB-1 and/or ErbB3 (aka HER3). In a particular embodiment, the EGFR inhibitor is EGFR specific (ErbB1, HER1). In another embodiment the EGFR inhibitor is ErbB3 specific (HER3). In yet another embodiment, the EGFR inhibitor targets EGFR with somatic mutations of the EGFR gene. These mutations can be small deletions that affect amino acids 747 through 750 or point mutations (most commonly a replacement of leucine by arginine at codon 858 [L858R]). In addition, or alternative, EGFR inhibitor can inhibit EGFR T790M. In a specific embodiment, EGFR inhibitor can inhibit a wild type EGFR.

For example, EGFR inhibitor can be selected from the group consisting of erlotinib, gefitinib, lapatinib, canertinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, panitumumab, matuzumab, pertuzumab, nimotuzumab, zalutumumab, icotinib, afatinib and cetuximab, and pharmaceutically acceptable salt thereof.

According to the present disclosure, gefitinib, cetuximab and erlotinib are particularly preferred EGFR inhibitors that can be combined with ceritinib.

Erlotinib (marketed in Tarceva®, Roche, Basel, Switzerland) is an EGFR inhibitor disclosed in WO 96/30347 as example 20 with formula

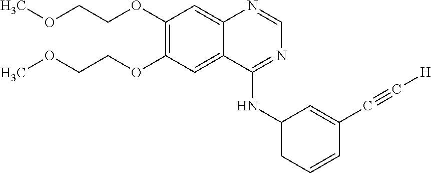 [SMILES: C#CC1=CC(Nc2ncnc3cc(OCCOC)c(OCCOC)cc23)CC=C1]

═N-(3-ethynylphenyl)-6,7-bis-(2-methoxyethoxy)quinazolin-4-amine, or a pharmaceutically acceptable salt thereof.

Gefitinib (marketed in Iressa®, AstraZeneca) was for example disclosed in WO96/33980, Example 1 with the structure

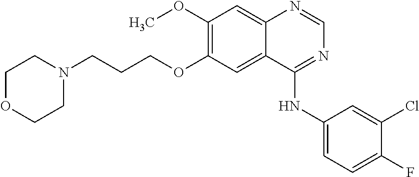 [SMILES: COc1cc2ncnc(Nc3ccc(F)c(Cl)c3)c2cc1OCCCN1CCOCC1]

═N-(3-chloro-4-fluorophenyl)-7-methoxy-6-[3-(morpholin-4-yl)propoxy]quinazolin-4-amine, or a pharmaceutically acceptable salt thereof. This compound or its pharmaceutically acceptable salts are especially preferred in the embodiments of the present disclosure.

Another EGFR inhibitor, lapatinib (marketed in Tykerb® (USA), Tyverb® (EP), GlaxoSmithKline) was disclosed in U.S. Pat. No. 6,391,874, U.S. Pat. No. 7,157,466, U.S. Pat. No. 6,828,320, US

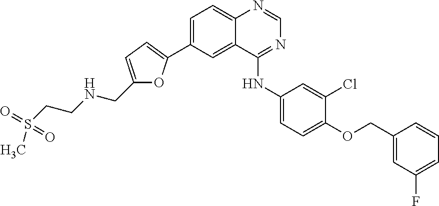 [SMILES: CS(=O)(=O)CCNCc1ccc(-c2ccc3ncnc(Nc4ccc(OCc5cccc(F)c5)c(Cl)c4)c3c2)o1]

═N-[3-chloro-4-(3-fluorobenzyloxy)phenyl]-6-{5-[4-(methylsulfonyl)-2-azabutyl]-2-furyl} quinazolin-4-amine, or a pharmeceutically acceptable salt or prodrug thereof, see e.g. WO9935146 (Example 29).

Canertinib (Pfizer) (e.g. used as dihydrochloride)), or N-[4-[(3-Chloro-4-fluorophenyl)amino]-7-(3-morpholin-4-ylpropoxy)quinazolin-6-yl]prop-2-enamide, or a pharmaceutically acceptable salt or prodrug thereof, was disclosed in WO2000031048 with the formula.

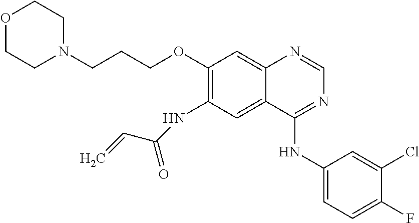 [SMILES: C=CC(=O)Nc1cc2c(Nc3ccc(F)c(Cl)c3)ncnc2cc1OCCCN1CCOCC1]

Pelitinib (Wyeth, owned by Pfizer) with the name (2E)-N-[4-[(3-Chloro-4-fluorophenyl)amino]-3-cyano-7-ethoxy-6-quinolinyl]-4-(dimethylamino)-2-butenamide, and formula

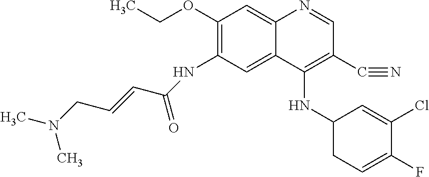 [SMILES: CCOc1cc2ncc(C#N)c(NC3C=C(Cl)C(F)=CC3)c2cc1NC(=O)/C=C/CN(C)C]

### see WO2005028443 (Example 20)

Neratinib (Pfizer Inc.), (2E)-N-[4-[[3-chloro-4-[(pyridin-2-yl)methoxy]phenyl]amino]-3-cyano-7-ethoxyquinolin-6-yl]-4-(dimethylamino)but-2-enamide, or a pharmaceutically acceptable salt or prodrug thereof with formula

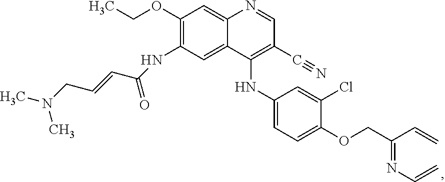 [SMILES: C=C/N=C(\C=C)COc1ccc(Nc2c(C#N)cnc3cc(OCC)c(NC(=O)/C=C/CN(C)C)cc23)cc1Cl]

see e.g. WO2005028443 Example 2.

Another EGFR inhibitor used in combination with ceritinib can be (R,E)-N-(7-chloro-1-(1-(4-(dimethylamino)but-2-enoyl)azepan-3-yl)-1H-benzo[d]imidazol-2-yl)-2-methylisonicotinamide (compound A)

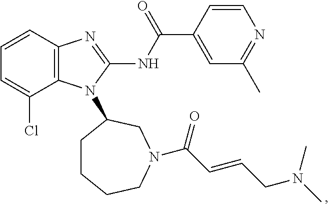 [SMILES: Cc1cc(C(=O)Nc2nc3cccc(Cl)c3n2[C@@H]2CCCCN(C(=O)/C=C/CN(C)C)C2)ccn1]

Another EGFR inhibitor, panitumumab (marketed as VECTIBIX®(US), Amgen) was disclosed in U.S. Pat. No. 6,235,883 and U.S. Pat. No. 7,807,798.

Another EGFR inhibitor, pertuzumab (marketed as PERJETA® (US), Genentech Inc.) was disclosed in WO200100244.

Another EGFR inhibitor, matuzumab (EMD 72000, Merk Serono), was disclosed in clinical trials studies (NCT00111839 and NCT00215644).

Another EGFR inhibitor, zalutumumab (developed by Genmab), was disclosed in clinical trials studies (NCT00093041) and Bastholt et al. Radiother. Oncol. 2007, 85(1), 24-28.

Another EGFR inhibitor, icotinib (approved in China under the name of Conmana, Zhejiang Bata Pharma Ltd.) was disclosed in WO2003082830 (example 15), with formula

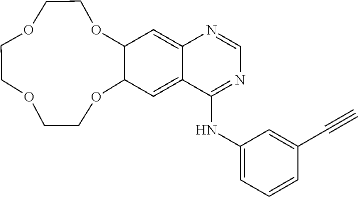 [SMILES: C#Cc1cccc(Nc2ncnc3c2=CC2OCCOCCOCCOC2C=3)c1]

=3-Ethylnyl-phenyl-(7,8,10,11,13,14-hexahydro-6,9,12,15-tetraoxa-1,3-diazacyclododeca[b]naphthalene-4-yl)-amine or a pharmaceutically acceptable salt thereof.

Another EGFR inhibitor, nimotuzumab (THERALOC® (EU), Oncoscience AG) was disclosed Grosse et al. J. Cell. Biochem. 1992, 49(2), 157-165 and Bartels et al. Future Oncol. 2009, 5(9), 1349-1361.

Another EGFR inhibitor, afatinib (marketed as GILOTRIF® (US), GIOTRIF® (EU), Boehringer Ingelheim Pharmaceuticals) was disclosed in U.S. Pat. No. 8,735,409B2

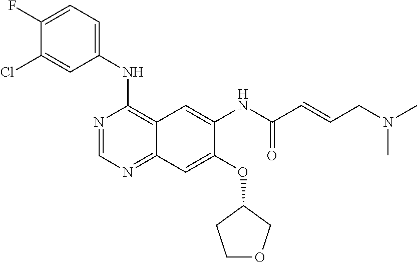 [SMILES: CN(C)C/C=C/C(=O)Nc1cc2c(Nc3ccc(F)c(Cl)c3)ncnc2cc1O[C@H]1CCOC1]

═(S,E)-N-(4-((3-chloro-4-fluorophenyl)amino)-7-((tetrahydrofuran-3-yl)oxy)quinazolin-6-yl)-4-(dimethylamino)but-2-enamide or a pharmaceutically acceptable salt thereof.

Among the possible EGFR inhibitors, also antibodies may be mentioned, e.g. Cetuximab (Erbitux®) (ImClone Systems, Bristol-Myers Squibb and Merck KgaA) which is a chimeric (mouse/human) monoclonal antibody, active as an epidermal growth factor receptor (EGFR) inhibitor, which can be administered e.g. intravenously.

In one embodiment, the EGFR inhibitor is an isolated antibody or fragment thereof to a HER3 receptor comprising 1, 2, 3, 4, 5, or 6 CDRs calculated by Kabat or Chothia of any of the antibodies shown in Table 1.

In one example, the EGFR inhibitor is an isolated antibody or fragment thereof comprising a heavy chain CDR3 selected from the group consisting of SEQ ID NO: 4, SEQ ID NO: 10, SEQ ID NO: 22, SEQ ID NO: 28, SEQ ID NO: 40, SEQ ID NO: 46, SEQ ID NO: 58, SEQ ID NO: 64, SEQ ID NO: 76, SEQ ID NO: 82, SEQ ID NO: 94, SEQ ID NO: 100, SEQ ID NO: 112, SEQ ID NO: 118, SEQ ID NO: 130, SEQ ID NO: 136, SEQ ID NO: 148, SEQ ID NO: 166, SEQ ID NO: 184, SEQ ID NO: 202, SEQ ID NO: 220, SEQ ID NO: 238, SEQ ID NO: 256, SEQ ID NO: 274, SEQ ID NO: 292, SEQ ID NO: 310, SEQ ID NO: 328, SEQ ID NO: 346, and SEQ ID NO: 364. Examples of isolated HER3 antibody or fragment include:

a VH comprising SEQ ID NO: 15 and a VL comprising SEQ ID NO: 14, or an amino acid sequence with 97-99 percent identity thereof;
a VH comprising SEQ ID NO: 33 and a VL comprising SEQ ID NO: 32, or an amino acid sequence with 97-99 percent identity thereof;
a VH comprising SEQ ID NO: 51 and a VL comprising SEQ ID NO: 50, or an amino acid sequence with 97-99 percent identity thereof;
a VH comprising SEQ ID NO: 69 and a VL comprising SEQ ID NO: 68, or an amino acid sequence with 97-99 percent identity thereof;
a VH comprising SEQ ID NO: 87 and a VL comprising SEQ ID NO: 86, or an amino acid sequence with 97-99 percent identity thereof;
a VH comprising SEQ ID NO: 105 and a VL comprising SEQ ID NO: 104, or an amino acid sequence with 97-99 percent identity thereof;
a VH comprising SEQ ID NO: 123 and a VL comprising SEQ ID NO: 122, or an amino acid sequence with 97-99 percent identity thereof
a VH comprising SEQ ID NO: 141 and a VL comprising SEQ ID NO: 140, or an amino acid sequence with 97-99 percent identity thereof;
a VH comprising SEQ ID NO: 159 and a VL comprising SEQ ID NO: 158, or an amino acid sequence with 97-99 percent identity thereof;
a VH comprising SEQ ID NO: 177 and a VL comprising SEQ ID NO: 176, or an amino acid sequence with 97-99 percent identity thereof;
a VH comprising SEQ ID NO: 195 and a VL comprising SEQ ID NO: 194, or an amino acid sequence with 97-99 percent identity thereof;
a VH comprising SEQ ID NO: 213 and a VL comprising SEQ ID NO: 212, or an amino acid sequence with 97-99 percent identity thereof;
a VH comprising SEQ ID NO: 231 and a VL comprising SEQ ID NO: 230, or an amino acid sequence with 97-99 percent identity thereof;
a VH comprising SEQ ID NO: 249 and a VL comprising SEQ ID NO: 248, or an amino acid sequence with 97-99 percent identity thereof;
a VH comprising SEQ ID NO: 267 and a VL comprising SEQ ID NO: 266, or an amino acid sequence with 97-99 percent identity thereof;
a VH comprising SEQ ID NO: 285 and a VL comprising SEQ ID NO: 284, or an amino acid sequence with 97-99 percent identity thereof
a VH comprising SEQ ID NO: 303 and a VL comprising SEQ ID NO: 302, or an amino acid sequence with 97-99 percent identity thereof;
a VH comprising SEQ ID NO: 321 and a VL comprising SEQ ID NO: 320, or an amino acid sequence with 97-99 percent identity thereof;
a VH comprising SEQ ID NO: 339 and a VL comprising SEQ ID NO: 338, or an amino acid sequence with 97-99 percent identity thereof;
a VH comprising SEQ ID NO: 357 and a VL comprising SEQ ID NO: 356, or an amino acid sequence with 97-99 percent identity thereof; and
a VH comprising SEQ ID NO: 375 and a VL comprising SEQ ID NO: 374, or an amino acid sequence with 97-99 percent identity thereof.

In one example, the EGFR inhibitor is an antibody or fragment thereof that recognizes a conformational epitope of a HER3 receptor comprising amino acid residues 265-277, and 315 within domain 2 and amino acid residues 571, 582-584, 596-597, 600-602, and 609-615 within domain 4 of the HER3 receptor of SEQ ID NO: 1, the sequence SEQ ID NO: 1 being as defined in WO2013/084148, and wherein the antibody or fragment thereof blocks both ligand-dependent and ligand-independent signal transduction. In alternative, the EGFR inhibitor is an antibody or fragment thereof that comprises a heavy chain variable region CDR1 of SEQ ID NO: 128; CDR2 of SEQ ID NO: 129; CDR3 of SEQ ID NO: 130; and a light chain variable region CDR1 of SEQ ID NO: 131; CDR2 of SEQ ID NO: 132; and CDR3 of SEQ ID NO: 133. Possible variant of the antibody or the fragment thereof are those that recognize a conformational epitope of a HER3 receptor as defined above and comprises at least one variable region CDR1 of SEQ ID NO: 128; CDR2 of SEQ ID NO: 129; or CDR3 of SEQ ID NO: 130; or at least one light chain variable region CDR1 of SEQ ID NO: 131; CDR2 of SEQ ID NO: 132; or CDR3 of SEQ ID NO: 133. A preferred EGFR inhibitor is an antibody or fragment thereof comprising the sequences of MOR10703 as defined in Table 1 (Antibody A). A process of preparing the antibody was described in WO2013/084148. Ceritinib or a pharmaceutically acceptable salt thereof can also be combined with an antibody comprising the sequences of MOR10703 as defined in Table 1 (Antibody A), or fragment thereof, and cetuximab. The Antibody A and cetuximab combined can significantly improve the efficacy of ceritinib. The triple combination is especially efficacious in the treatment of NSCLC.

An EGFR inhibitor (HER3 inhibitor), which can be combined with ceritinib, can be an antibody that comprises a heavy chain variable region CDR1 of SEQ ID NO: 128. An EGFR inhibitor can also be an antibody that comprises a heavy chain variable region CDR2 of SEQ ID NO: 129. The antibody can comprise a heavy chain variable region CDR3 of SEQ ID NO: 130.

An EGFR inhibitor (HER3 inhibitor), which can be combined with ceritinib, can be an antibody that comprises a light chain variable region CDR1 of SEQ ID NO: 131. In the same or alternative embodiment, an EGFR inhibitor can be an antibody that comprises a light chain variable region CDR2 of SEQ ID NO: 132. An EGFR inhibiting antibody can also comprise a light chain variable region CDR3 of SEQ ID NO: 133. Further combinations of said CDRs are contemplated herein.

TABLE 1

| | |
|---|---|
| SEQ ID NUMBER | Ab region |

MOR09823

| | | |
|---|---|---|
| SEQ ID NO: 2(Kabat) | HCDR1 | SYAMS |
| SEQ ID NO: 3(Kabat) | HCDR2 | VTGAVGRTYYPDSVKG |
| SEQ ID NO: 4(Kabat) | HCDR3 | WGDEGFDI |
| SEQ ID NO: 5(Kabat) | LCDR1 | PASQGISNWLA |
| SEQ ID NO: 6(Kabat) | LCDR2 | GASSLQS |
| SEQ ID NO: 7(Kabat) | LCDR3 | QQYSSFPTT |
| SEQ ID NO: 8(Chothia) | HCDR1 | GFTFSSY |
| SEQ ID NO: 9(Chothia) | HCDR2 | GAVGR |
| SEQ ID NO: 10(Chothia) | HCDR3 | WGDEGFDI |
| SEQ ID NO: 11(Chothia) | LCDR1 | SQGISNW |
| SEQ ID NO: 12(Chothia) | LCDR2 | GAS |
| SEQ ID NO:(Chothia) 13 | LCDR3 | YSSFPT |
| SEQ ID NO: 14 | VL | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIK |
| SEQ ID NO: 15 | VH | QVQLVESGGGLVQPGGSLRLSCAASGFTFSSYAMSWVRQAPGKGLEWVSV |
| | | TGAVGRTYYPDSVKGRFTISRDNSKNTLYLQMNSLRAEDTAVYYCARWGD |
| | | EGFDIIWGQGTLVTVSS |
| SEQ ID NO: 16 | DNA VL | GATATCCAGATGACCCAGAGCCCGTCTAGCCTGAGCGCGAGCGTGGGTGATCGTG |
| | | TGACCATTACCTGCAGAGCGAGCCAGGGTATTTCTAATTGGCTGGCTTGGTACCA |
| | | GCAGAAACCAGGTAAAGCACCGAAACTATTAATTTATGGTGCTTCTTCTTTGCAA |
| | | AGCGGGGTCCCGTCCCGTTTTAGCGGCTCTGGATCCGGCACTGATTTTACCCTGA |
| | | CCATTAGCAGCCTGCAACCTGAAGACTTTGCGGTTTATTATTGCCAGCAGTATTC |
| | | TTCTTTTCCTACTACCTTTGGCCAGGGTACGAAAGTTGAAATTAAA |
| SEQ ID NO: 17 | DNA VH | CAGGTGCAATTGGTGGAAAGCGGCGGCGGCCTGGTGCAACCGGGCGGCAGCCTGC |
| | | GTCTGAGCTGCGCGGCCTCCGGATTTACCTTTAGCAGCTATGCGATGAGCTGGGT |
| | | GCGCCAAGCCCCTGGGAAGGGTCTCGAGTGGGTGAGCGTTACTGGTGCTGTTGGT |
| | | CGTACTTATTATCCTGATTCTGTTAAGGGTCGTTTTACCATTTCACGTGATAATT |
| | | CGAAAAACACCCTGTATCTGCAAATGAACAGCCTGCGTGCGGAAGATACGGCCGT |
| | | GTATTATTGCGCGCGTTGGGGTGATGAGGGTTTTGATATTTGGGGCCAAGGCACC |
| | | CTGGTGACGGTTAGCTCA |
| SEQ ID NO: 18 | Light Kappa | DIQMTOSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| SEQ ID NO: 19 | Heavy IgG1 | QVQLVESGGGLVQPGGSLRLSCAASGFTFSSYAMSWVRQAPGKGLEWVSV |
| | | TGAVGRTYYPDSVKGPFTISRDNSKNTLYLQMNSLRAEDTAVYYCARWGD |
| | | EGFDIWGQGTLVTVSSASTKGPSVFPLAPSSKSTSGGTAALGCLVKDYFP |
| | | EPVTVSWNSGALTSGVHTFPAVLQSSGLYSLSSVVTVPSSSLGTQTYICN |
| | | VNHKPSNTKVDKRVEPKSCDKTHTCPPCPAPELLGGPSVFLFPPKPKDTL |
| | | MISRTPEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQVYTL |
| | | PEPREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |

MOR09824

| | | |
|---|---|---|
| SEQ ID NO: 20(Kabat) | HCDR1 | SYAMS |
| SEQ ID NO: 21(Kabat) | HCDR2 | VISAWGHVKYYADSVKG |
| SEQ ID NO: 22(Kabat) | HCDR3 | WGDEGEFI |
| SEQ ID NO: 23(Kabat) | LCDR1 | RASQGISNWLA |
| SEQ ID NO: 24(Kabat) | LCDR2 | GASSLQS |
| SEQ ID NO: 25(Kabat) | LCDR3 | QQYSSFPTT |
| SEQ ID NO: 26(Chothia) | HCDR1 | GFTFSSY |
| SEQ ID NO: 27(Chothia) | HCDR2 | SAWGHV |
| SEQ ID NO: 28 | HCDR3 | WGDEGFDI |
| (Chothia) | | |
| SEQ ID NO: 29(Chothia) | LCDR1 | SQGISNW |
| SEQ ID NO: 30(Chothia) | LCDR2 | GAS |
| SEQ ID NO: 31(Chothia) | LCDR3 | YSSFPT |
| SEQ ID NO: 32 | VL | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIK |
| SEQ ID NO: 33 | VH | QVQLVESGGGLVQPGGSLRLSCAASGFTFSSYAMSWVRQAPGKGLEWVSV |
| | | ISAWGHVKYYADSVKGRFTISRDNSKNTLYLQMNSLRAEDTAVYYCARWG |
| | | DEGFDIWGQGTLVTVSS |
| SEQ ID NO: 34 | DNA VL | GATATCGAGATGACCCAGAGCCCGTCTAGCCTGAGCGCGAGCGTGGGTGATCGTG |
| | | TGACCATTACCTGCAGAGCGAGCCAGGGTATTTCTAATTGGCTGGCTTGGTACCA |
| | | GCAGAAACCAGGTAAAGCACCGAAACTATTAATTTATGGTGCTTCTTCTTTGCAA |
| | | AGCGGGGTCCCGTCCCGTTTTAGCGGCTCTGGATCCGGCACTGATTTTACCCTGA |
| | | CCATTAGCAGCCTGCAACCTGAAGACTTTGCGGTTTATTATTGCCAGCAGTATTC |
| | | TTCTTTTCCTACTACCTTTGGCCAGGGTACGAAAGTTGAAATTAAA |
| SEQ ID NO: 35 | DNA VH | CAGGTGCAATTGGTGGAAAGCGGCGGCGGCCTGGTGCAACCGGGCGGCAGCCTGC |
| | | GTCTGAGCTGCGCGGCCTCCGGATTTACCTTTAGCAGCTATGCGATGAGCTGGGT |
| | | GCGCCAAGCCCCTGGGAAGGGTCTCGAGTGGGTGAGCGTTATTTCTGCTTGGGGT |
| | | CATGTTAAGTATTATGCTGATTCTGTTAAGGGTCGTTTTACCATTTCACGTGATA |
| | | ATTCGAAAAACACCCTGTATCTGCAAATGAACAGCCTGCGTGCGGAAGATACGGC |
| | | CGTGTATTATTGCGCGCGTTGGGGTGATGAGGGTTTTGATATTTGGGGCCAAGGC |
| | | ACCCTGGTGACGGTTAGCTCA |
| SEQ ID NO: 36 | Light Kappa | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIKRTVAAPSVFIFPESDEQLKSGTASVVCLLNNFYPREAKVQWKV |
| | | DNALQSGNSQESVTEQDSKDSTYSLSSTLTLSKADYEKHKVYACEVTGQG |
| | | LSSPVTKSFNRGEC |
| SEQ ID NO: 37 | Heavy IgG1 | QVQLVESGGGLVQPGGSLRLSCAASGFTFSSYAMSWVRQAPGKGLEWVSV |
| | | ISAWGHVKYYADSVKGRFTISRDNSKNTLYLQMNSLRAEDTAVYYCARWG |
| | | DEGFDIWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDT |
| | | LMISCTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |

MOR09825

| | | |
|---|---|---|
| SEQ ID NO: 38(Kabat) | HCDR1 | SYAMS |
| SEQ ID NO: 39(Kabat) | HCDR2 | AINSQGKSTYYADSVKG |
| SEQ ID NO: 40(Kabat) | HCDR3 | WGDEGFIDI |
| SEQ ID NO: 41(Kabat) | LCDR1 | RASQGISNWLA |
| SEQ ID NO: 42(Kabat) | LCDR2 | GASSLQS |
| SEQ ID NO: 43(Kabat) | LCDR3 | QQYSSFPTT |
| SEQ ID NO: 44(Chothia) | HCDR1 | GFTFSSY |
| SEQ ID NO: 45(Chothia) | HCDR2 | NSQGKS |
| SEQ ID NO: 46(Chothia) | HCDR3 | WGDEGFDI |
| SEQ ID NO: 47(Chothia) | LCDR1 | SQGISNW |
| SEQ ID NO: 48(Chothia) | LCDR2 | GAS |
| SEQ ID NO: 49(Chothia) | LCDR3 | YSSFPT |
| SEQ ID NO: 50 | VL | DIOMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIK |
| SEQ ID NO: 51 | VH | QVOLVESGGGLVQPGGSLRLSCAASGFTFSSYAMSWVRQAPGKGLEWVSA |
| | | INSQGKSTYYADSVKGRFTISRDNSKNTLYLQMNSLRAEDTAVYYCARWG |
| | | DEGFDIWGQGTLVTVSS |
| SEQ ID NO: 52 | DNA VL | GATATCCAGATGACCCAGAGCCCGTCTAGCCTGAGCGCGAGCGTGGGTGATCGTG |
| | | TGACCATTACCTGCAGAGCGAGCCAGGGTATTTCTAATTGGCTGGCTTGGTACCA |
| | | GCAGAAACCAGGTAAAGCACCGAAACTATTAATTTATGGTGCTTCTTCTTTGCAA |
| | | AGCGGGGTCCCGTCCCGTTTTAGCGGCTCTGGATCCGGCACTGATTTTACCCTGA |
| | | CCATTAGCAGCCTGCAACCTGAAGACTTTGCGGTTTATTATTGCCAGCAGTATTC |
| | | TTCTTTTCCTACTACCTTTGGCCAGGGTACGAAAGTTGAAATTAAA |
| SEQ ID NO: 53 | DNA VH | CAGGTGCAATTGGTGGAAAGCGGCGGCGGCCTGGTGCAACCGGGCGGCAGCCTGC |
| | | GTCTGAGCTGCGCGGCCTCCGGATTTACCTTTAGCAGCTATGCGATGAGCTGGGT |
| | | GCGCCAAGCCCCTGGGAAGGGTCTCGAGTGGGTGAGCGCTATTAATTCTCAGGGT |
| | | AAGTCTACTTATTATGCTGATTCTGTTAAGGGTCGTTTTACCATTTCACGTGATA |
| | | ATTCGAAAAACACCCTGTATCTGCAAATGAACAGCCTGCGTGCGGAAGATACGGC |
| | | CGTGTATTATTGCGCGCGTTGGGGTGATGAGGGTTTTGATATTTGGGGCCAAGGC |
| | | ACCCTGGTGACGGTTAGCTCA |
| SEQ ID NO: 54 | Light Kappa | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| SEQ ID NO: 55 | Heavy IgG1 | QVQLVESGGGLVQPGGSLRLSCAASGFTFSSYAMSWVRQAPGKGLEWVSA |
| | | INSQGKSTYYADSVKGRFTISRDNSKNTLYLQMNSLRAEDTAVYYCARWG |
| | | DEGFDIWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |

MOR09974

| | | |
|---|---|---|
| SEQ ID NO: 56(Kabat) | HCDR1 | SYAMSS |
| SEQ ID NO: 57(Kabat) | HCDR2 | VINPSGNFTNYADSVKG |
| SEQ ID NO: 58(Kabat) | HCDR3 | WGDEGFDI |
| SEQ ID NO: 59(Kabat) | LCDR1 | RASQGISNWLA |
| SEQ ID NO: 60(Kabat) | LCDR2 | GASSLQS |
| SEQ ID NO: 61(Kabat) | LCDR3 | QQYSSFPTT |
| SEQ ID NO: 62(Chothia) | HCDR1 | GFTFSSY |
| SEQ ID NO: 63(Chothia) | HCDR2 | NPSGNF |
| SEQ ID NO: 64(Chothia) | HCDR3 | WGDEGFDI |
| SEQ ID NO: 65(Chothia) | LCDR1 | SQGISNW |
| SEQ ID NO: 66(Chothia) | LCDR2 | GAS |
| SEQ ID NO: 67(Chothia) | LCDR3 | YSSFPT |
| SEQ ID NO: 68 | VL | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIK |
| SEQ ID NO: 69 | VH | QVQLVESGGGLVQPGGSLRSCAASGFTFSSYAMSQVRQAPGKGLEWVSV |
| | | INPSGNFTNYADSVKGRFTISRDNSKNTLYQMNSLRAEDTAVYYCARWG |
| | | DEGFDIWQGTLVTVSS |
| SEQ ID NO: 70 | DNA VL | GATATCCAGATGACCCAGAGCCCGTCTAGCCTGAGCGCGAGCGTGGGTGATCGTG |
| | | TGACCATTACCTGCAGAGCGAGCCAGGGTATTTCTAATTGGCTGGCTTGGTACCA |
| | | GCAGAAACCAGGTAAAGCACCGAAACTATTAATTTATGGTGCTTCTTCTTTGCAA |
| | | AGCGGGGTCCCGTCCCGTTTTAGCGGCTCTGGATCCGGCACTGATTTTACCCTGA |
| | | CCATTAGCAGCCTGCAACCTGAAGACTTTGCGGTTTATTATTGCCAGCAGTATTC |
| | | TTCTTTTCCTACTACCTTTGGCCAGGGTACGAAAGTTGAAATTAAA |
| SEQ ID NO: 71 | DNA VH | CAGGTGCAATTGGTGGAAAGCGGCGGCGGCCTGGTGCAACCGGGCGGCAGCCTGC |
| | | GTCTGAGCTGCGCGGCCTCCGGATTTACCTTTAGCAGCTATGCGATGAGCTGGGT |
| | | GCGCCAAGCCCCTGGGAAGGGTCTCGAGTGGGTGAGCGTTATTAATCCTTCTGGT |
| | | AATTTTACTAATTATGCTGATTCTGTTAAGGGTCGTTTTACCATTTCACGTGATA |
| | | ATTCGAAAAACACCCTGTATCTGCAAATGAACAGCCTGCGTGCGGAAGATACGGC |
| | | CGTGTATTATTGCGCGCGTTGGGGTGATGAGGGTTTTGATATTTGGGGCCAAGGC |
| | | ACCCTGGTGACGGTTAGCTA |
| SEQ ID NO: 72 | Light Kappa | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHAVYACEVTHQG |
| | | LSSPVTKSFNRGEC |
| SEQ ID NO: 73 | Heavy IgG1 | QVQLVESGGGLVQPGGSLRLSCAASGFTFSSYAMSWVRQAPGKGLEWVSV |
| | | INPSGNFTNYADSVKGRFTISRDNSANTLYLQMNSLRAEDTAVYYCARWG |
| | | DEGFDIWGQGTLVTVSSASTKGPSVFPLAPSSKSTSGGTAALGCLVADYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPEPKAT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |

MOR10452

| | | |
|---|---|---|
| SEQ ID NO: 74(Kabat) | HCDR1 | SYAMS |
| SEQ ID NO: 75(Kabat) | HCDR2 | NTSPIGYTYYAGSVKG |
| SEQ ID NO: 76(Kabat) | HCDR3 | WGDEGFDI |
| SEQ ID NO: 77(Kabat) | LCDR1 | RASQGISNWLA |
| SEQ ID NO: 78(Kabat) | LCDR2 | GASSLQS |
| SEQ ID NO: 79(Kabat) | LCDR3 | QQYSSFPTT |
| SEQ ID NO: 80(Chothia) | HCDR1 | GFTFSSY |
| SEQ ID NO: 81(Chothia) | HCDR2 | SPIGY |
| SEQ ID NO: 82(Chothia) | HCDR3 | WGDEGFDI |
| SEQ ID NO: 83(Chothia) | LCDR1 | SQGISNW |
| SEQ ID NO: 84(Chothia) | LCDR2 | GAS |
| SEQ ID NO: 85(Chothia) | LCDR3 | YSSFPT |
| SEQ ID NO: 86 | VL | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIK |
| SEQ ID NO: 87 | VH | QVQLVESGGGLVQPGGSLALSCAASGFTFSSYAMSWVROAPGKGLEWVSN |
| | | TSPIGYTYYAGSVKGRFTISRDNSKNTLYLQMNSLRAEDTAVYYCARWGD |
| | | EGFDIWGQGTLVTVSS |
| SEQ ID NO: 88 | DNA VL | GATATCCAGATGACCCAGAGCCCGTCTAGCCTGAGCGCGAGCGTGGGTGATCGTG |
| | | TGACCATTACCTGCAGAGCGAGCCAGGGTATTTCTAATTGGCTGGCTTGGTACCA |
| | | GCAGAAACCAGGTAAAGCACCGAAACTATTAATTTATGGTGCTTCTTCTTTGCAA |
| | | AGCGGGGTCCCGTCCCGTTTTAGCGGCTCTGGATCCGGCACTGATTTTACCCTGA |
| | | CCATTAGCAGCCTGCAACCTGAAGACTTTGCGGTTTTATTATGCCAGCAGTATTC |
| | | TTCTTTTCCTACTACCTTTGGCCAGGGTACGAAAGTTGAAATTAAA |
| SEQ ID NO: 89 | DNA VH | CAGGTGCAATTGGTGGAAAGCGGCGGCGGCCTGGTGCAACCGGGCGGCAGCCTGC |
| | | GTCTGAGCTGCGCGGCCTCCGGATTTACCTTTAGCAGCTATGCGATGAGCTGGGT |
| | | GCGCCAAGCCCCTGGGAAGGGTCTCGAGTGGGTGAGCAATACTTCTCCTATTGGT |
| | | TATACTTATTATGCTGGTTCTGTTAAGGGTCGTTTTACCATTTCACGTGATAATT |
| | | CGAAAAACACCCTGTATCTGCAAATGAACAGCOTGCGTGCGGAAGATACGGCCGT |
| | | GTATTATTGCGCGCGTTGGGGTGATGAGGGTTTTGATATTTGGGGCCAAGGCACC |
| | | CTGGTGACGGTTAGCTCA |
| SEQ ID NO: 90 | Light Kappa | DIQMTQSPSSLSASVGDRVTITCRASQGISNWLAWYQQKPGKAPKLLIYG |
| | | ASSLQSGVPSRFSGSGSGTDFTLTISSLQPEDFAVYYCQQYSSFPTTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEA |
| SEQ ID NO: 91 | Heavy Chain | QVQLVESGGGLVQPGGSLRLSCAASGFTFSSYAMSWVRQAPGKGLEWVSN |
| | (only VH and Ch1 | TSPIGYTYYAGSVKGRFTISRDNSKNTLYLQMNSLRAEDTAVYYCARWGD |
| | domains) | EGFDIWGQGTLVTVSSASTKGPSVFPLAPSSKSTSGGTAALGCLVKDYFP |
| | | EPVTVSKNSGALTSGVHTFPAVLQSSGLYSLSSVVTVPSSSLGTQTYICN |
| | | VNHKPSNTKVDKKVEPKS |

MOR10701

| | | |
|---|---|---|
| SEQ ID NO: 92(Kabat) | HCDR1 | SYAMSS |
| SEQ ID NO: 93(Kabat) | HCDR2 | VTGAVGRSTYYPDSVKG |
| SEQ ID NO: 94(Kabat) | HCDR3 | WGDEGFDI |
| SEQ ID NO: 95(Kabat) | LCDR1 | RASQGISNWLA |
| SEQ ID NO: 96(Kabat) | LCDR2 | GASSLQS |
| SEQ ID NO: 97(Kabat) | LCDR3 | QQYSSFPTT |
| SEQ ID NO: 98(Chothia) | HCDR1 | GFTFSSY |
| SEQ ID NO: 99(Chothia) | HCDR2 | GAVGRS |
| SEQ ID NO: 100(Chothia) | HCDR3 | WGDEGFDI |
| SEQ ID NO: 101(Chothia) | LCDR1 | SQGISNW |
| SEQ ID NO: 102(Chothia) | LCDR2 | GAS |
| SEQ ID NO: 103(Chothia) | LCDR3 | YSSFPT |
| SEQ ID NO: 104 | VL | DIQMTQSPSSLSASVGDRVTITCRASQGISNWLAWYQQKPGKAPKLLIYG |
| | | ASSLQSGVPSRFSGSGSGTIFTLTISSLQPEDFATYYCQQYSSFPTTFGQ |
| | | GTKVEIK |
| SEQ ID NO: 105 | VH | EVQLLESGGGLVQPGGSLRLSCAASGFTFSSYAMSWVRQAPGKGLEWVSV |
| | | TGAVGRSTYYPDSVKGRFTISPDNSKNTLYLQMNSLRAEDTAVYYCARWG |
| | | DEGFDIWGQGTLVTVSS |
| SEQ ID NO: 106 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTAGTGCCAGCAGTACAG |
| | | CAGCTTCCCCACCACCTTCGGCCAGGGCACCAAGGTGGAAATCAAG |
| SEQ ID NO: 107 | DNA VH | GAGGTGCATTTGCTGGAAAGCGGCGGAGGCCTGGTGCAGCCTGGCGGCAGCCTGA |
| | | GACTGTCTTGCGCCGCCAGCGGCTTCACCTTCAGCACCTACGCCATGAGCTGGGT |
| | | CCCCCAGGCCCCTGGCAAGGGACTGGAATGGGTGTCCGTGACAGGCGCCGTGGGC |
| | | AGAAGCACCTACTACCCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 108 | Light Kappa | DIQMTQSPSSLSASVGDRVTITCRASQGISNWLAWYQQKPGKAPKLLIYG |
| | | ASSLQSGVPSRFSGSGSGTDFTLTISSLQPEDFATYYCQQYSSFPTTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSNRGEC |
| SEQ ID NO: 109 | Heavy IgG1 | EVQLLESGGGLVQPGGSLPLSCAASGFTFSSYAMSWVRQAPGKGLEWVSV |
| | | TGAVGRSTYYPDSVKGTFTISRDNSKNTLYLQMNSLRAEDTAVYYCARWG |
| | | DEGFDIWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |

MOR10702

| | | |
|---|---|---|
| SEQ ID NO: 110(Kabat) | HCDR1 | SYAMS |
| SEQ ID NO: 111(Kabat) | HCDR2 | VISAWGHVKYYADSVKG |
| SEQ ID NO: 112(Kabat) | HCDR3 | WGDEGFDI |
| SEQ ID NO: 113(Kabat) | LCDR1 | RASQGISNWLA |
| SEQ ID NO: 114(Kabat) | LCDR2 | GASSLQS |
| SEQ ID NO: 115(Kabat) | LCDR3 | QQYSSFPTT |
| SEQ ID NO: 116(Chothia) | HCDR1 | GFTFSSY |
| SEQ ID NO: 117(Chothia) | HCDR2 | SAWGHV |
| SEQ ID NO: 118 | HCDR3 | WGDEGFDI |
| (Chothia) | | |
| SEQ ID NO: 119(Chothia) | LCDR1 | SQGISNW |
| SEQ ID NO: 120(Chothia) | LCDR2 | GAS |
| SEQ ID NO: 121(Chothia) | LCDR3 | YSSFPT |
| SEQ ID NO: 122 | VL | DIQMTQSPSSLSASVGDRVTITCRASQGISNWLAWYQQKPGKAPKLLIYG |
| | | ASSLQSGVPSRFSGSGSGTDFTLTISSLQPEDFATYYCQQYSSFPTTFGQ |
| | | GTKVEIK |
| SEQ ID NO: 123 | VH | EVQLLESGGGLVQPGGSLRLSCAASGFTFSSYAMSWVRQAPGKGLEWVSV |
| | | ISAWGHVKYYADSVKGRFTISRDNSKNTLYLQMNSLRAEDTAVYYCARWG |
| | | DEGFDIWGQGTLVTVSS |
| SEQ ID NO: 124 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 125 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGTGATCAGCGCCTGGGGC |
| | | CACGTGAAGTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCATCAGCTCA |
| SEQ ID NO: 126 | Light Kappa | DIQMTQSPSSLSASVGDRVTITCRASQGISNWLAWYQQKPGKAPKLLIYG |
| | | ASSLQSGVPSRFSGSGSGTDFTLTISSLQPEDFATYYCOQYSSFPTTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| SEQ ID NO: 127 | Heavy IgG1 | EVQLLESGGGLVQPGGSLRLSCAASGFTFSSYAMSWVRQAPGKGLEWVSV |
| | | ISAWGHVKYYADSVKGRFTISRDNSKNTLYLQMNSLRAEDTAVYYCARWG |
| | | DEGFDIWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGPSVFLFPPKTKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |

MOR10703

| | | |
|---|---|---|
| SEQ ID NO: 128(Kabat) | HCDR1 | SYAMS |
| SEQ ID NO: 129(Kabat) | HCDR2 | AINSQGKSTYYADSVKG |
| SEQ ID NO: 130(Kabat) | HCDR3 | WGDEGFDI |
| SEQ ID NO: 131(Kabat) | LCDR1 | RASQGISNWLA |
| SEQ ID NO: 132(Kabat) | LCDR2 | GASSLQS |
| SEQ ID NO: 133(Kabat) | LCDR3 | QQYSSFPTT |
| SEQ ID NO: 134(Chothia) | HCDR1 | GFTFSSY |
| SEQ ID NO: 135(Chothia) | HCDR2 | NSQGKS |
| SEQ ID NO: 136(Chothia) | HCDR3 | WGDEGFDI |
| SEQ ID NO: 137(Chothia) | LCDR1 | SQGISNW |
| SEQ ID NO: 138(Chothia) | LCDR2 | GAS |
| SEQ ID NO: 139(Chothia) | LCDR3 | YSSFPT |
| SEQ ID NO: 140 | VL | DIQMTQSPSSLSASVGDRVTITCRASQGISNWLAWYQQKPGKAPKLLIYG |
| | | ASSLQSGVPSRFSGSGSGTDFTLTISSLQPEDFATYYCQQYSSFPTTFGQ |
| | | GTKVEIK |
| SEQ ID NO: 141 | VH | EVQLLESGGGLVQPGGSLRLSCAASGFTFSSYAMSWVRQAPGKGLEWVSA |
| | | INSQGKSTYYADSVKGRFTISRDNSKNTLYLQMNSLRAEDTAVYYCARWG |
| | | DEGFDIWGQGTLVTVSS |
| SEQ ID NO: 142 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 143 | DNA VH | GAGGTGCAATTGCTGGAAAGCGGCGGAGGCCTGGTGCAGCCTGGCGGCAGCCTGA |
| | | GACTGTCTTGCGCCGCCAGOGGCTTCACCTTCAGCAGCTACGCCATGAGCTGGGT |
| | | CCGCCAGGCCCCTGGCAAGGGACTGGAATGGGTGTCCGCCATCAACAGCCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 144 | Light Kappa | DIQMTQSPSSLSASVGDRVTITCRASQGISNWLAWYQQKPGKAPKLLIYG |
| | | ASSLQSGVPSRFSGSGSGTDFTLTISSLQPEDFATYYCQQYSSFPTTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| SEQ ID NO: 145 | Heavy IgG1 | EVQLLESGGGLVQPGGSLRLSCAASGFTFSSYAMSWVRQAPGKGLEWVSA |
| | | INSQGKSTYYADSVKGRFTISRDNSKNTLYLQMNSLRAEDTAVYYCARWG |
| | | DEGFDIWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYIC |
| | | NVNHKPSNTKVDKRVEPKSCDKTHTCPPCPAPELLGGESVFLFPPKPKDT |
| | | LMISRTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQVYT |
| | | LPPSREEMTKNQVSLTCLVKGFYPSDIAVEWESMGQPENNYKTTPPVLDS |
| | | DGSFFLYSKLTVDKSRWQQGNVFSCSVMHEALHNHYTQKSLSLSPGK |

M0R10703 N52S

| | | |
|---|---|---|
| SEQ ID NO: 146(Kabat) | HCDR1 | SYAMS |
| SEQ ID NO: 147(Kabat) | HCDR2 | AISSQGKSTYYADSVKG |
| SEQ ID NO: 148(Kabat) | HCDR3 | WGDEGFDI |
| SEQ ID NO: 149(Kabat) | LCDR1 | RASQGISNWLA |
| SEQ ID NO: 150(Kabat) | LCDR2 | GASSLQS |
| SEQ ID NO: 151(Kabat) | LCDR3 | QOYSSFPTT |
| SEQ ID NO: 152(Chothia) | HCDR1 | GFTFSSY |
| SEQ ID NO: 153(Chothia) | HCDR2 | SSQGKS |
| SEQ ID NO: 154(Chothia) | HCDR3 | WGDEGFDI |
| SEQ ID NO: 155(Chothia) | LCDR1 | SQGISNW |
| SEQ ID NO: 156(Chothia) | LCDR2 | GAS |
| SEQ ID NO: 157(Chothia) | LCDR3 | YSSFPT |
| SEQ ID NO: 158 | VL | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIK |
| SEQ ID NO: 159 | VH | EVQLLESGGGLVQPGGSLRLSCAASGFTFSSYAMSWVRQAPGKGLEWVSAISSQG |
| | | KSTYYADSVKGRFTISRDNSKNTLYLQMNSLRAEDTAVYYCARWGDEGFDIWGQG |
| | | TLVTVSS |
| SEQ ID NO: 160 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 161 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGCCATCAGCAGCCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 162 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 163 | Heavy IgG1 | EVQLLESGGGLVQPGGSLRLSCAASGFTFSSYAMSWVRQAPGKGLEWVSAISSQG |
| | | KSTYYADSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |

MOR10703 N52G

| | | |
|---|---|---|
| SEQ ID NO: 164(Kabat) | HCDR1 | SYAMS |
| SEQ ID NO: 165(Kabat) | HCDR2 | AIGSQGKSTYYADSVKG |
| SEQ ID NO: 166(Kabat) | HCDR3 | WGDEGFDI |
| SEQ ID NO: 167(Kabat) | LCDR1 | RASQGISNWLA |
| SEQ ID NO: 168(Kabat) | LCDR2 | GASSLQS |
| SEQ ID NO: 169(Kabat) | LCDR3 | QQYSSFPTT |
| SEQ ID NO: 170(Chothia) | HCDR1 | GFTFSSY |
| SEQ ID NO: 171(Chothia) | HCDR2 | GSQGKS |
| SEQ ID NO: 172(Chothia) | HCDR3 | WGDEGFDI |
| SEQ ID NO: 173(Chothia) | LCDR1 | SQGISNW |
| SEQ ID NO: 174(Chothia) | LCDR2 | GAS |
| SEQ ID NO: 175(Chothia) | LCDR3 | YSSFPT |
| SEQ ID NO: 176 | VL | DIQMTQPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIK |
| SEQ ID NO: 177 | VH | EVQLLESGGGLVQPGGSLRLSCAASGFTFSSYAMSWVRQAPGKGLEWVSAIGSQG |
| | | KSTYYADSVKGRFTISRDNSKNTLYLQMNSLRAEDTAVYYCARWGDEGFDIWGQG |
| | | TLVTVSS |
| SEQ ID NO: 178 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 179 | DNAVH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGCCATCGGCAGCCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 180 | Light Kappa | DIQMTQPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 181 | Heavy IgG1 | EVQLLESGGGLVQPGGSLRLSCAASGFTFSSYAMSWVRQAPGKGLEWVSAIGSQG |
| | | KSTYYADSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVYHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |

MOR10703 N525_S52aN

| | | |
|---|---|---|
| SEQ ID NO: 182(Kabat) | HCDR1 | SYAMS |
| SEQ ID NO: 183(Kabat) | HCDR2 | AISNQGKSTYYADSVKG |
| SEQ ID NO: 184(Kabat) | HCDR3 | WGDEGFDI |
| SEQ ID NO: 185(Kabat) | LCDR1 | RASQGISNWLA |
| SEQ ID NO: 186(Kabat) | LCDR2 | GASSLQS |
| SEQ ID NO: 187(Kabat) | LCDR3 | QQYSSFPTT |
| SEQ ID NO: 188(Chothia) | HCDR1 | GFTFSSY |
| SEQ ID NO: 189(Chothia) | HCDR2 | SNQGKS |
| SEQ ID NO: 190(Chothia) | HCDR3 | WGDEGFDI |
| SEQ ID NO: 191(Chothia) | LCDR1 | SQGISNW |
| SEQ ID NO: 192(Chothia) | LCDR2 | GAS |
| SEQ ID NO: 193(Chothia) | LCDR3 | YSSFPT |
| SEQ ID NO: 194 | VL | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIK |
| SEQ ID NO: 195 | VH | EVQLLESGGGLVQPGGSLRLSCAASGFTFSSYAMSWVRQAPGKGLEWVSAISNQG |
| | | KSTYYADSVKGRFTISRDNSKNTLYLQMNSLRAEDTAVYYCARWGDEGFDIWGQG |
| | | TLVTVSS |
| SEQ ID NO: 196 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 197 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGCCATCAGCAACCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACGTGCCAGATGGGGCGACGAGGGCTTCGACATCTGGGGCCAGGGC |
| | | ACCCTGGTCACCGTCAGCTCA |
| SEQ ID NO: 198 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 199 | Heavy IgG1 | EVQLLESGGGLVQPGGSLRLSCAASGFTFSSYAMSWVRQAPGKGLEWVSAISNQG |
| | | KSTYYADSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |

M0R10703 A50V_N52S

| | | |
|---|---|---|
| SEQ ID NO: 200(Kabat) | HCDR1 | SYAMS |
| SEQ ID NO: 201(Kabat) | HCDR2 | VISSQGKSTYYADSVKG |
| SEQ ID NO: 202(Kabat) | HCDR3 | WGDEGFDI |
| SEQ ID NO: 203(Kabat) | LCDR1 | RASQGISNWLA |
| SEQ ID NO: 204(Kabat) | LCDR2 | GASSLQS |
| SEQ ID NO: 205(Kabat) | LCDR3 | QQYSSFPTT |
| SEQ ID NO: 206(Chothia) | HCDR1 | GFTFSSY |
| SEQ ID NO: 207(Chothia) | HCDR2 | SSQGKS |
| SEQ ID NO: 208(Chothia) | HCDR3 | WGDEGFDI |
| SEQ ID NO: 209(Chothia) | LCDR1 | SQGISNW |
| SEQ ID NO: 210(Chothia) | LCDR2 | GAS |
| SEQ ID NO: 211(Chothia) | LCDR3 | YSSFPT |
| SEQ ID NO: 212 | VL | DIQMTQSPSSLSASVGDRVTITCRASQGISNWLAWYQQKPGKAPKLLIYGASSLQ |
| | | SGVPSRFSGSGSGTDFTLTISSLQPEDFATYYCQOYSSFPTTFGQGTKVEIK |
| SEQ ID NO: 213 | VH | EVQLLESGGGLVQPGGSLRLSCAASGFTFSSYAMSWVRQAPGKGLEWVSVISSQG |
| | | KSTYYADSVKGRFTISRDNSKNTLYLQMNSLRAEDTAVYYCARWGDEGFDIWGQG |
| | | TLVTVSS |
| SEQ ID NO: 214 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 215 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGTCATCAGCAGCCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 216 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNKFYPREAKVQWKVDKALQSGNSQESVTE |
| | | QDSKDSTYSLSSTLTLSKADYEKHKVYACEVTHQGLSSPVTKSFNRGEC |
| SEQ ID NO: 217 | Heavy IgG1 | EVQLLESGGGLVQPGGSLRLSCAASGFTFSSYAMSWVRQAPGKGLEWVSVISSQG |
| | | KSTYYADSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |

M0R10703 A50V_N52G

| | | |
|---|---|---|
| SEQ ID NO: 218(Kabat) | HCDR1 | SYAMS |
| SEQ ID NO: 219(Kabat) | HCDR2 | VIGSQCKSTYYADSVI1G |
| SEQ ID NO: 220(Kabat) | HCDR3 | WGDEGFDI |
| SEQ ID NO: 221(Kabat) | LCDR1 | RASQGISNWLA |
| SEQ ID NO: 222(Kabat) | LCDR2 | GASSLQS |
| SEQ ID NO: 223(Kabat) | LCDR3 | QQYSSFPTT |
| SEQ ID NO: 224(Chothia) | HCDR1 | GFTFSSY |
| SEQ ID NO: 225(Chothia) | HCDR2 | GSQGKS |
| SEQ ID NO: 226(Chothia) | HCDR3 | WGDEGEDI |
| SEQ ID NO: 227(Chothia) | LCDR1 | SQGISNW |
| SEQ ID NO: 228(Chothia) | LCDR2 | GAS |
| SEQ ID NO: 229(Chothia) | LCDR3 | YSSFPT |
| SEQ ID NO: 230 | VL | DIQMTQSPSSLSASVGDRVTITCRASQGISWWLAWYQQKPGKAPKLLIYGASSLQ |
| | | SGVPSRFSGSGSGTDFTLTISSLQPEDFATYYCQQYSSFPTTFGQGTKVEIK |
| SEQ ID NO: 231 | VH | EVQLLESGGGLVQPGGSLPLSCAASGFTFSSYAMSWVRQAPGKGLEWVSVIGSQG |
| | | KSTYYADSVKGRFTISRDNSKNTLYLQMNSLRAEDTAVYYCARWGDEGFDIWGQG |
| | | TLVTVSS |
| SEQ ID NO: 232 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 233 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCCCCAGGCCCCTGGCAAGGGACTGGAATGGGTGTCCGTCATCGGCAGCCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 234 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFATYYCOQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 235 | Heavy IgG1 | EVQLLESGGGLVQPGGSLRLSCAASGFTFSSYAMSWVRQAPGKGLEWVSVIGSQG |
| | | KSTYYADSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMEEALHNHYTQKS |
| | | LSLSPGK |

MOR10703 S52aA

| | | |
|---|---|---|
| SEQ ID NO: 236(Kabat) | HCDR1 | SYAMS |
| SEQ ID NO: 237(Kabat) | HCDR2 | AINAQGKSTYYADSVKG |
| SEQ ID NO: 238(Kabat) | HCDR3 | WGDEGFDI |
| SEQ ID NO: 239(Kabat) | LCDR1 | RASQGISNWLA |
| SEQ ID NO: 240(Kabat) | LCDR2 | GASSLQS |
| SEQ ID NO: 241(Kabat) | LCDR3 | QQYSSFPTT |
| SEQ ID NO: 242(Chothia) | HCDR1 | GFTFSSY |
| SEQ ID NO: 243(Chothia) | HCDR2 | NAQGKS |
| SEQ ID NO: 244(Chothia) | HCDR3 | WGDEGFDI |
| SEQ ID NO: 245(Chothia) | LCDR1 | SQGISNW |
| SEQ ID NO: 246(Chothia) | LCDR2 | GAS |
| SEQ ID NO: 247(Chothia) | LCDR3 | YSSFPT |
| SEQ ID NO: 248 | VL | DIQMTQSPSSLSASVGDRVTITCRASQGISNWLAWYDQKPGKAPKLLIYGASSLQ |
| | | SGVPSRFSGSGSGTDFTLTISSLQPEDFATYYCQGYSSFPTTFGQGTKVEIK |
| SEQ ID NO: 249 | VH | EVQLLESGGGLVQPGGSLRLSCAASGFTRSSYAMSWVRQAPGKGLEWVSAINAQG |
| | | KSTYYADSVKGRFTISRDNSKNTLQMNSLRAEDTAVYYCARWGDEGFDIWGQG |
| | | TLVTVSS |
| SEQ ID NO: 250 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGGAGATTCAGCGGCAGCGGCTCCGGCACCGACTTCACCCTGA |
| | | CCATCAGCAGCCTGCAGCCCGAGGACTTCGCCACCTACIACTGCCAGCAGTACAG |
| | | CAGCTTCCCCACCACCTTCGGCCAGGGCACCAAGGTGGAAATCAAG |
| SEQ ID NO: 251 | DNA VH | GAGGTGCAATTGCTGakkAGCGGCGGAGGCCTGGTGCAGCCTGGCGGCAGCCTGA |
| | | GACTGTCTTGCGCCGCCAGCGGCTTCAGCTTCAGCAGCTACGCCATGAGCTGGGT |
| | | CCGCCAGGCCCCTGGCAAGGGAGTGGAATGGGTGTCCGCCATCAACGCCCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 252 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 253 | Heavy IgG1 | EVQLLESGGGLVQPGGSLRLSCAASGFTFSSYAMSWVRQAPGKGLEWVSAINAQG |
| | | KSTYYADSVKGRFTISRDNSKNTLYLQMNSLRAEDTAVYYCARWGDEGFDIWGOG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNMKPSNTKVDKRVEPKSC |
| | | DKTHTCPPCPAPELLGGPSVFLFPPKPKDTLMISRTPEVTCVVVDVSMEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYQOKS |
| | | LSLSPGK |

M0R10703 S52aT

| | | |
|---|---|---|
| SEQ ID NO: 254(Kabat) | HCDR1 | SYAMS |
| SEQ ID NO: 255(Kabat) | HCDR2 | AINTQGKSTYYADSVKG |
| SEQ ID NO: 256(Kabat) | HCDR3 | WGDEGFDI |
| SEQ ID NO: 257(Kabat) | LCDR1 | RASQGISNWLA |
| SEQ ID NO: 258(Kabat) | LCDR2 | GASSLQS |
| SEQ ID NO: 259(Kabat) | LCDR3 | QQYSSFPTT |
| SEQ ID NO: 260(Chothia) | HCDR1 | GFTFSSY |
| SEQ ID NO: 261(Chothia) | HCDR2 | NTQGKS |
| SEQ ID NO: 262(Chothia) | HCDR3 | WGDEGFDI |
| SEQ ID NO: 263(Chothia) | LCDR1 | SQGISNW |
| SEQ ID NO: 264(Chothia) | LCDR2 | GAS |
| SEQ ID NO: 265(Chothia) | LCDR3 | YSSFPT |
| SEQ ID NO: 266 | VL | DIQMTQSPSSLSASVGDRVTITCRASQGISNWLAWYQQKPGKATKLLIYGASSLQ |
| | | SGVPSRFSGSGSGTDFTLTISSLQPEDFATYYCQQYSSFPTTFGQGTKVEIK |
| SEQ ID NO: 267 | VH | EVQLLESGGGLVQPGGSLRLSCAASGFTFSSYAMSWVPQAPGKGLEWVSAINTQG |
| | | KSTYYADSVKGPFTISRDNSKNTLYLQMNSLRAEDTAVYYCARWGDEGFDIWGQG |
| | | TLVTVSS |
| SEQ ID NO: 268 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 269 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCCCCAGGCCCCTGGCAAGGGACTGGAATGGGTGTCCGCCATCAACACCCAGGGC |
| | | AAGAGCACCTACTACGCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 270 | Light Kappa | DIQMTQSPSSLSAVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 271 | Heavy IgG1 | EVQLLESGGGLVQPGGSLRLSCRASGFTFSSYAMSWVRQAPGKGLEWVSAINTQG |
| | | KSTYYADSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |

MOR10701 R55S

| | | |
|---|---|---|
| SEQ ID NO: 272(Kabat) | HCDR1 | SYAMS |
| SEQ ID NO: 273(Kabat) | HCDR2 | VTGAVGSSTYYPDSVKG |
| SEQ ID NO: 274(Kabat) | HCDR3 | WGDEGFDI |
| SEQ ID NO: 275(Kabat) | LCDR1 | RASQGISNWLA |
| SEQ ID NO: 276(Kabat) | LCDR2 | GASSLQS |
| SEQ ID NO: 277(Kabat) | LCDR3 | QQYSSFPTT |
| SEQ ID NO: 278(Chothia) | HCDR1 | GFTFSSY |
| SEQ ID NO: 279(Chothia) | HCDR2 | GAVGSS |
| SEQ ID NO: 280(Chothia) | HCDR3 | WGDEGFDI |
| SEQ ID NO: 281(Chothia) | LCDR1 | SQGISNW |
| SEQ ID NO: 282(Chothia) | LCDR2 | GAS |
| SEQ ID NO: 283(Chothia) | LCDR3 | YSSFPT |
| SEQ ID NO: 284 | VL | DIQMTQSPSSLSASVGDRVTITCRASQGISNWLAWYQQKPGKAPKLLIYGASSLQ |
| | | SGVPSRFSGSGSGDFTLTISSLQPEDFATYYCQQYSSFPTTFGQGTKVEIK |
| SEQ ID NO: 285 | VH | EVQLLESGGGLVQPGGSLRLSCAASGFTFSSYAMSWVRQAPGKGLEWVSVTGAVG |
| | | SSTYYPDSVKGRFTISRDNSKNTLYLQMNSLRAEDTAVYYCARWGDEGFDIWGQG |
| | | TLVTVSS |
| SEQ ID NO: 286 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 287 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGTGACAGGCGCCGTGGGC |
| | | AGCAGCACCTACTACCCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 288 | Light Kappa | DIQMTOSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 289 | Heavy IgG1 | EVQLLESGGGLVQPGGSLRLSCAASGFTFSSYAMSWVRQAPGKGLEWVSVTGAVG |
| | | SSTYYPDSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |

MOR10701 R55G

| | | |
|---|---|---|
| SEQ ID NO: 290(Kabat) | HCDR1 | SYAMS |
| SEQ ID NO: 291(Kabat) | HCDR2 | VTGAVGGSTYYPDSVKG |
| SEQ ID NO: 292(Kabat) | HCDR3 | WGDEGFDI |
| SEQ ID NO: 293(Kabat) | LCDR1 | RASQGISNWLA |
| SEQ ID NO: 294(Kabat) | LCDR2 | GASSLQS |
| SEQ ID NO: 295(Kabat) | LCDR3 | QQYSSFPTT |
| SEQ ID NO: 296(Chothia) | HCDR1 | GFTFSSY |
| SEQ ID NO: 297(Chothia) | HCDR2 | GAVGGS |
| SEQ ID NO: 298(Chothia) | HCDR3 | WGDEGFDI |
| SEQ ID NO: 299(Chothia) | LCDR1 | SQGISNW |
| SEQ ID NO: 300(Chothia) | LCDR2 | GAS |
| SEQ ID NO: 301(Chothia) | LCDR3 | YSSFPT |
| SEQ ID NO: 302 | VL | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIK |
| SEQ ID NO: 303 | VH | EVQLLESGGGLVQPGGSLRLSCAASGFTFSSYAMSWVRQAPGKGLEWVSVTGAVG |
| | | GSTYYPDSVKGRFTISRDNSKNTLYLQMNSLRAEDTAVYYCARWGDEGFDIWGQG |
| | | TLVTVSS |
| SEQ ID NO: 304 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGCTATCA |
| | | GCAGAAGCCCGGCAAGGCCCCCAAGCTGCTGATCTACGCCCCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 305 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGTGACAGGCGCCGTGGGC |
| | | GGATGCACCTACTACCCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 306 | Light Kappa | DIQMTQSPSSLSASVGDRVTITCRASQGISNWLANYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 307 | Heavy IgG1 | EVQLLESGGGLVQPGGSLRLSCAASGFTFSSYAMSWVRQAPGKGLEWVSVTGAVG |
| | | GSTYYPDSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSPFLYSKLTVDKSRWQQGNVFSCSVMHEALHNHYTQKS |
| | | LSLSPGK |

MOR10701 R55K

| | | |
|---|---|---|
| SEQ ID NO: 308(Kabat) | HCDR1 | SYAMS |
| SEQ ID NO: 309(Kabat) | HCDR2 | VTGAVGKSTYYPDSVKG |
| SEQ ID NO: 310(Kabat) | HCDR3 | WGDEGFDI |
| SEQ ID NO: 311(Kabat) | LCDR1 | RASQGISNWLA |
| SEQ ID NO: 312(Kabat) | LCDR2 | GASSLQS |
| SEQ ID NO: 313(Kabat) | LCDR3 | QQYSSFPTT |
| SEQ ID NO: 314(Chothia) | HCDR1 | GFTFSSY |
| SEQ ID NO: 315(Chothia) | HCDR2 | GAVGKS |
| SEQ ID NO: 316(Chothia) | HCDR3 | WGDEGFDI |
| SEQ ID NO: 317(Chothia) | LCDR1 | SQGISNW |
| SEQ ID NO: 318(Chothia) | LCDR2 | GAS |
| SEQ ID NO: 319(Chothia) | LCDR3 | YSSFPT |
| SEQ ID NO: 320 | VL | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIK |
| SEQ ID NO: 321 | VH | EVQLLESGGGLVQPGGSLRLSCAASGFTFSSYAMSWVRQAPGKGLEWVSVTGAVG |
| | | KSTYYPDSVKGRFTISRDNSKNTLYLQMNSLRAEDTAVYYCARWGDEGFDIWGQG |
| | | TLVTVSS |
| SEQ ID NO: 322 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 323 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGTGACAGGCGCCGTGGGC |
| | | AAAAGCACCTACTACCCCGACAGCGTGAAGGGCCGGTTCACCATCAGCCGGGACA |
| | | ACAGCAAGAACACCCTGTACCTGCAGATGAACAGCCTGCGGGCCGAGGACACCGC |
| | | CGTGTACTACTGTGCCAGATGGGGCGACGAGGGCTTCGACATCTGGGGCCAGGGC |
| | | ACCCTGGTCACCGTCAGCTCA |
| SEQ ID NO: 324 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 325 | Heavy IgG1 | EVQLLESGGGLVQPGGSLRLSCAASGFTFSSYAMSWVRQAPGKGLEWVSVTGAVG |
| | | KSTYYPDSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPVVLSSDGSFFLYSKLTVDKSRWQQGNVFSCSVHBEALHNHYTQKS |
| | | LSLSPGK |

MOR10701 deletion S56

| | | |
|---|---|---|
| SEQ ID NO: 326(Kabat) | HCDR1 | SYAMS |
| SEQ ID NO: 327(Kabat) | HCDR2 | VTGAVGRTYYPDSVKG |
| SEQ ID NO: 328(Kabat) | HCDR3 | WGDEGFDI |
| SEQ ID NO: 329(Kabat) | LCDR1 | RASQGISNWLA |
| SEQ ID NO: 330(Kabat) | LCDR2 | GASSLQS |
| SEQ ID NO: 331(Kabat) | LCDR3 | QQYSSFPTT |
| SEQ ID NO: 332(Chothia) | HCDR1 | GFTFSSY |
| SEQ ID NO: 333(Chothia) | HCDR2 | GAVGRT |
| SEQ ID NO: 334(Chothia) | HCDR3 | WGDEGFDI |
| SEQ ID NO: 335(Chothia) | LCDR1 | SQGISNW |
| SEQ ID NO: 336(Chothia) | LCDR2 | GAS |
| SEQ ID NO: 337(Chothia) | LCDR3 | YSSFPT |
| SEQ ID NO: 338 | VL | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIK |
| SEQ ID NO: 339 | VH | EVQLLESGGGLVQPGGSLRLSCAASGFTFSSYAMSWVRQAPGKGLEWVSVTGAVG |
| | | RTYYPDSVKGRFTISRDNSKNTLYLQMNSLRAEDTAVYYCARWGDEGFDIWGQGT |
| | | LVTVSS |
| SEQ ID NO: 340 | DNA VL | GATATCCAGATGACCCAGAGCCCCAGCAGCCTGAGCGCCAGCGTGGGCGACAGAG |
| | | TGACCATCACCTGTCGGGCCAGCCAGGGCATCAGCAACTGGCTGGCCTGGTATCA |
| | | GCAGAAGCCCGGCAAGGCCCCCAAGCTGCTGATCTACGGCGCCAGCTCCCTGCAG |
| | | AGCGGCGTGCCAAGCAGATTCAGCGGCAGCGGCTCCGGCACCGACTTCACCCTGA |
| | | CCATCAGCAGCCTGCAGCCCGAGGACTTCGCCACCTACTACTGCCAGCAGTACAG |
| | | CAGCTTCCCCACCACCTTCGGCCAGGGCACCAAGGTGGAAATCAAG |
| SEQ ID NO: 341 | DNA VH | GAGGTGCAATTGCTGGAAAGCGGCGGAGGCCTGGTGCAGCCTGGCGGCAGCCTGA |
| | | GACTGTCTTGCGCCGCCAGCGGCTTCACCTTCAGCAGCTACGCCATGAGCTGGGT |
| | | CCGCCAGGCCCCTGGCAAGGGACTGGAATGGGTGTCCGTGACAGGCGCCGTGGGC |
| | | AGAACCTACTACCCCGACAGCGTGAAGGGCCGGTTCACCATCAGCCGGGACAACA |
| | | GCAAGAACACCCTGTACCTGCAGATGAACAGCCTGCGGGCCGAGGACACCGCCGT |
| | | GTACTACTGTGCCAGATGGGGCGACGAGGGCTTCGACATCTGGGGCCAGGGCACC |
| | | CTGGTCACCGTCAGCTCA |
| SEQ ID NO: 342 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFATYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 343 | Heavy IgG1 | EVQLLESGGGLVQPGGSLRLSCAASGFTFSSYAMSWVRQAPGKGLEWVSVTGAVG |
| | | RTYYPDSVKGRFTISRDNSKNTLYLQMNSLRAEDTAVYYCARWGDEGFDIWGQGT |
| | | LVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |

MOR12609

| | | |
|---|---|---|
| SEQ ID NO: 344(Kabat) | HCDR1 | SYAMS |
| SEQ ID NO: 345(Kabat) | HCDR2 | VINGLGYTTFYADSVKG |
| SEQ ID NO: 346(Kabat) | HCDR3 | WGDEGFDI |
| SEQ ID NO: 347(Kabat) | LCDR1 | RASQGISNWLA |
| SEQ ID NO: 348(Kabat) | LCDR2 | GASSLQS |
| SEQ ID NO: 349(Kabat) | LCDR3 | QQYSSFPTT |
| SEQ ID NO: 350(Chothia) | HCDR1 | GFTFSSY |
| SEQ ID NO: 351(Chothia) | HCDR2 | NGLGYT |
| SEQ ID NO: 352(Chothia) | HCDR3 | WGDEGFDI |
| SEQ ID NO: 353(Chothia) | LCDR1 | SQGISNW |
| SEQ ID NO: 354(Chothia) | LCDR2 | GAS |
| SEQ ID NO: 355(Chothia) | LCDR3 | YSSFPT |
| SEQ ID NO: 356 | VL | DIQMTQSPSSLSASVGDRVTITCRASQGISNWLAWYQQKPGKAPKLLIYGASSLQ |
| | | SGVPSRFSGSGSGTDFTLTISSLQPEDFAVYYCQQYSSFPTTFGQGTKVEIK |
| SEQ ID NO: 357 | VH | QVQLVESGGGLVQPGGSLRLSCAASGFTFSSYAMSWVRQAPGKGLEWVSVINGLG |
| | | YTTFYADSVKGRFTISRDNSKNTLYLQMNSLRAEDTAVYYCARWGDEGFDIWGQG |
| | | TLVTVSS |
| SEQ ID NO: 358 | DNA VL | GATATCCAGATGACCCAGAGCCCGTCTAGCCTGAGCGCGAGCGTGGGTGATCGTG |
| | | TGACCATTACCTGCAGAGCGAGCCAGGGTATTTCTAATTLGGTGGCTTGGTACCA |
| | | GCAGAAACCAGGTAAAGCACCGAAACTATTAATTTATGGTGCTTCTTCTTTGCAA |
| | | AGCGGGGTCCCGTCCCGTTTTAGCGGCTCTGGATCCGGCACTGATTTTACCCTGA |
| | | CCATTAGCAGCCTGCAACCTGAAGACTTTGCGGTTTATTATTGCCAGCAGTATTC |
| | | TTCTTTTCCTACTACCTTTGGCCAGGGTACGAAAGTTGAAATTAAA |
| SEQ ID NO: 359 | DNA VH | CAGGTGCAATTGGTGGAAAGCGGCGGCGGCCTGGTGCAACCGGGCGGCAGCCTGC |
| | | GTCTGAGCTGCGCGGCCTCCGGATTTACCTTTAGCAGCTATGCGATGAGCTGGGT |
| | | GCGCCAAGCCCCTGGGAAGGGTCTCGAGTGGGTGAGCGTTATTAATGGTCTTGGT |
| | | TATACTACTTTTTATGCTGATTCTGTTAAGGGTCGTTTTACCATTTCACGTGATA |
| | | ATTCGAAAAACACCCTGTATCTGCAAATGAACAGCCTGCGTGCGGAAGATATGGC |
| | | CGTGTATTATTGCGCGCGTTGGGGTGATGAGGGTTTTGATATTTGGGGCCAAGGC |
| | | ACCCTGGTGACGGTTAGCTCA |
| SEQ ID NO: 360 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFAVYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 361 | Heavy IgG1 | QVQLVESGGGLVQPGGSLRLSCAASGFTFSSYAMSWVRQAPGKGLEWVSVINGLG |
| | | YTTFYADSVKGRFTISRDNSKNTLYLQMNSLRAEDTAVYYCARWGDEGFDIWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |

MOR12610

| | | |
|---|---|---|
| SEQ ID NO: 362(Kabat) | HCDR1 | SYAMS |
| SEQ ID NO: 363(Kabat) | HCDR2 | GTGPYGGTYYPDSVKG |
| SEQ ID NO: 364(Kabat) | HCDR3 | WGDEGFDI |
| SEQ ID NO: 365(Kabat) | LCDR1 | RASQGISNWLA |
| SEQ ID NO: 366(Kabat) | LCDR2 | GASSLQS |
| SEQ ID NO: 367(Kabat) | LCDR3 | QQYSSFPTT |
| SEQ ID NO: 368(Chothia) | HCDR1 | GFTFSSY |
| SEQ ID NO: 369(Chothia) | HCDR2 | GPYGG |
| SEQ ID NO: 370(Chothia) | HCDR3 | WGDEGFDI |
| SEQ ID NO: 371(Chothia) | LCDR1 | SQGISNW |
| SEQ ID NO: 372(Chothia) | LCDR2 | GAS |
| SEQ ID NO: 373(Chothia) | LCDR3 | YSSFPT |
| SEQ ID NO: 374 | VL | DIQMTQPSSLSASVGDRVTITCRASQGISNWLAWYQQKPGKAPKLLIYGASSLQ |
| | | SGVPSRFSGSGSGTDFTLTISSLQPEDFAVYYCQQYSSFPTTFGQGTKVEIK |
| SEQ ID NO: 375 | VH | QVQLVESGGGLVQPGGSLRLSCAASGFTFSSYAMSWVRQAPGKGLEWVSGTGPYG |
| | | GTYYPDSVKGRFTISRDNSKNTLYLQMNSLRAEDTAVYYCARWGDEGFDIWGQGT |
| | | LVTVSS |
| SEQ ID NO: 376 | DNA VL | GATATCCAGATGACCCAGAGCCCGTCTAGCCTGAGCGCGAGCGTGGGTGATCGTG |
| | | TGACCATTACCTGCAGAGCGAGCCAGGGTATTTCTAATTGGCTGGCTTGGTACCA |
| | | GCAGAAACCAGGTAAAGCACCGAAACTATTAATTTATGGTGCTTCTTCTTTGCAA |
| | | AGCGGGGTCCCGTCCCGTTTTAGCGGCTCTGGATCCGGCACTGATTTTACCCTGA |
| | | CCATTAGCAGCCTGCAACCTGAAGACTTTGCGGTTTATTATTGCCAGCAGTATTC |
| | | TTCTTTTCCTACTACCTTTGGCCAGGGTACGAAAGTTGAAATTAAA |
| SEQ ID NO: 377 | DNA VH | CAGGTGCAATTGGTGGAkAGCGGCGGCGGCCTGGTGCAACCGGGCGGCAGCCTGC |
| | | GTCTGAGCTGCGCGGCCTCCGGATTTACCTTTAGCAGCTATGCGATGAGCTGGGT |
| | | GCGCCAAGCCCCTGGGAAGGGTCTCGAGTGGGTGAGCGGTACTGGTCCTTATGGT |
| | | GGTACTTATTATCCTGATTCTGTTAAGGGTGOTTTTACCATTTCACGTGATAATT |
| | | CGAAAAACACCCTGTATCTGCAAATGAACAGCCTGCGTGCGGAAGATACGGCCGT |
| | | GTATTATTGCGCGCGTTGGGGTGATGAGGGTTTTGATATTTGGGGCCAAGGCACC |
| | | CTGGTGACGGTTAGCTCA |
| SEQ ID NO: 378 | Light Kappa | DIQMTQSPSSLSASVGDRVTITCRASQGISNWLAWYQQKPGKAPKLLIYGASSLQ |
| | | SGVPSRFSGSGSGTDFTLTISSLQPEDFAVYYCQQYSSFPTTFGQGTKVEIKRTV |
| | | AAPSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| SEQ ID NO: 379 | Heav IgG1 | QVQLVESGGGLVQPGGSLRLSCAASGFTFSSYAMSWVRQAPGKGLEWVSGTGPYG |
| | | GTYYPDSVKGRFTISRDNSKNTLYLQMNSLRAEDTAVYYCARWGDEGFDIWGQGT |
| | | LVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKRVEPKSCD |
| | | KTHTCPPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |

In accordance with the present disclosure the compounds in the pharmaceutical combination, components (I) ceritinib, or a pharmaceutically acceptable salt thereof, and (ii) an EGFR inhibitor, or a pharmaceutically acceptable salt thereof can be administered separately or together.

The pharmaceutical combination, according to the present disclosure, for use as a medicine, wherein ceritinib and the EGFR inhibitor are administered independently at the same time or separately within time intervals, wherein time intervals allow that the combination partners are jointly active.

The term “pharmaceutical combination” as used herein refers to a product obtained from mixing or combining in a non-fixed combination the active ingredients, e.g. (i) ceritinib, or a pharmaceutically acceptable salt thereof, and (ii) an EGFR inhibitor or a pharmaceutically acceptable salt thereof separately or together.

The term “combination” refers to formulations of the separate partners with or without instructions for combined use or to combination products. The combined compounds can be manufactured and/or formulated by the same or different manufacturers. The combination partners may thus be entirely separate pharmaceutical dosage forms or pharmaceutical compositions that are also sold independently of each other and where just instructions for their combined use are provided: (i) prior to release to physicians (e.g. in the case of a “kit of part” comprising the compound of the disclosure and the other therapeutic agent); (ii) by the physician themselves (or under the guidance of a physician) shortly before administration; (iii) the patient themselves by a physician or medical staff.

The term “non-fixed combination” means that the active ingredients, e.g. ceritinib and an EGFR tyrosine kinase inhibitor, are both administered separately or together, independently at the same time or separately within time intervals, wherein such administration provides therapeutically effective levels of the active ingredient in the subject in need. The latter also applies to cocktail therapy, e.g. the administration of three or more active ingredients. This term defines especially a “kit of parts” in the sense that the combination partners (i) ceritinib and (ii) EGFR inhibitor (and if present further one or more co-agents) as defined herein can be dosed independently of each other. Nevertheless, it is contemplated herein that ceritinib and an EGFR inhibitor could be administered in a reduced dose compared to the respective doses used when the drugs are used alone. Particularly this can be advantageous in case tolerability and drug related adverse events are problematic when using the compound. Drug dose reduction is such instances could help to keep the subject, e.g. patient, on the drug, while adding the combination partner. Overall, such approach bestows the clinical team with better flexibility as to the treatment options for the subject. One example of such combination where the reduced doses of either one, or both, of the drugs is administered is the combination of ceritinib and afatinib. It would be expected that gastrointestinal side effects, for example, diarrhea, could be adequately mitigated or at least reduced.

The present disclosure also includes the pharmaceutical combination according to the disclosure, wherein the EGFR inhibitor is erlotinib, gefitinib or cetuximab.

In another embodiment, the pharmaceutical combination according to the disclosure comprises as an EGFR inhibitor an isolated antibody or fragment thereof to a HER3 receptor comprising 1, 2, 3, 4, 5, or 6 CDRs calculated by Kabat or Chothia of any of the antibodies shown in Table 1.

A further embodiment of the disclosure provides a combination (e.g. combination product) comprising a quantity which is jointly therapeutically effective for the treatment of an ALK and/or EGFR mediated disease. The term “jointly therapeutically effective” means that the compounds show synergistic interaction when administered separately or together, independently at the same time or separately within time intervals, to treat a subject in need, such as a warm-blooded animal in particular a human.

The term “mediated disease” means that the condition or the disease lacks a definitive etiology, but is characterized by a common pathway leading to a medical disorder such as dysregulation, inflammation, immune response, cell growth, cell apoptosis, allergy.

It was shown that the combination of the present disclosure possesses beneficial therapeutic properties, e.g. synergistic interaction, strong in-vivo and in-vitro antitumor response, which can be used as a medicine. Its characteristics render it particularly useful for the treatment of cancer.

Suitable cancers that can be treated with the combination of the present disclosure include but are not limited to anaplastic large cell lymphoma (ALCL), neuroblastoma, lung cancer, non-small cell lung cancer (NSCLC). In a preferred embodiment, the cancer is NSCLC. In one embodiment, the cancer comprises wt EGFR. In another embodiment, the cancer comprises T790M EGFR.

A further embodiment of the disclosure relates to the pharmaceutical combination according to the present disclosure, wherein the ALK and/or EGFR mediated disease is NSCLC.

The combination according to the present disclosure can besides or in addition be administered especially for cancer therapy in combination with chemotherapy, radiotherapy, immunotherapy, surgical intervention, or in combination of these. Long-term therapy is equally possible as is adjuvant therapy in the context of other treatment strategies, as described above. Other possible treatments are therapy to maintain the patient's status after tumor regression, or even chemo-preventive therapy, for example in patients at risk.

The terms “treat”, “treating” or “treatment” of any disease or disorder refers to ameliorating the disease or disorder (e.g. slowing or arresting or reducing the development of the disease or at least one of the clinical symptoms thereof), to preventing or delaying the onset or development or progression of the disease or disorder. In addition those terms refers to alleviating or ameliorating at least one physical parameter including those which may not be discernible by the patient and also to modulating the disease or disorder, either physically (e.g. stabilization of a discernible symptom), physiologically (e.g. stabilization of a physical parameter), or both.

The term “treatment” comprises, for example, the therapeutic administration of the combination partners to a warm-blooded animal, in particular a human being, in need of such treatment with the aim to cure the disease or to have an effect on disease regression or on the delay of progression of a disease.

The term “subject in need” refers to a warm-blooded animal, in particular a human being that would benefit biologically, medically or in quality of life from the treatment.

The combination of ceritinib and EGFR inhibitor can be used to manufacture a medicament for an ALK and/or EGFR mediated disease as described above. Likewise the combination can be used in a method for the treatment of an ALK and/or an EGFR mediated disease, as described above, said method comprising administering an effective amount of a combination of (i) ceritinib, or a pharmaceutically acceptable salt thereof, and (ii) an EGFR inhibitor or a pharmaceutically acceptable salt thereof separately or together, to a subject in need thereof, according to the present disclosure.

For example, the term “jointly (therapeutically) active” may mean that the compounds may be given separately or sequentially (in a chronically staggered manner, especially a sequence specific manner) in such time intervals that they preferably, in the warm-blooded animal, especially human, to be treated, and still show a (preferably synergistic) interaction (joint therapeutic effect). A joint therapeutic effect can, inter alia, be determined by following the blood levels, showing that both compounds are present in the blood of the human to be treated at least during certain time intervals, but this is not to exclude the case where the compounds are jointly active although they are not present in blood simultaneously.

Subject or patient that can get the combination administered encompasses mammals and non-mammals. Examples of mammals include, but are not limited to, humans, chimpanzees, apes, monkeys, cattle, horses, sheep, goats, swine; rabbits, dogs, cats, rats, mice, guinea pigs, and the like. Examples of non-mammals include, but are not limited to, birds, fish and the like. In a most preferred embodiment, the subject or patient is human. It may be a human who has been diagnosed as in need of treatment for a disease or disorder disclosed herein.

The present disclosure also describes the method for the treatment of an ALK and/or an EGFR mediated disease, wherein the combination of (i) ceritinib, or a pharmaceutically acceptable salt thereof, and (ii) an EGFR inhibitor or a pharmaceutically acceptable salt thereof separately or together.

The present disclosure relates to a pharmaceutical composition comprising effective amounts of (i) ceritinib, or a pharmaceutically acceptable salt thereof, and (ii) an EGFR inhibitor, or a pharmaceutically acceptable salt thereof. The pharmaceutical composition can be prepared with a pharmaceutically acceptable carrier, which can be for example any suitable pharmaceutical excipient. The carrier includes any and all binders, fillers, solvents, dispersion media, coatings, surfactants, antioxidants, preservatives (e.g., antibacterial agents, antifungal agents), isotonic agents, absorption delaying agents, salts, drug stabilizers, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329; Remington: The Science and Practice of Pharmacy, 21st Ed. Pharmaceutical Press 2011; and subsequent versions thereof). Except insofar as any conventional carrier is incompatible with the active ingredient, its use in the therapeutic or pharmaceutical compositions is contemplated.

In accordance with the present disclosure the combination partners can be administered independently at the same time or separately within time intervals in separate unit dosage forms. The two therapeutic partners may be prepared in a manner known per se and are suitable for enteral, such as oral or rectal, topical and parenteral administration to subject in need thereof, including warm-blooded animal, in particular a human being. Suitable pharmaceutical compositions contain, e.g. from about 0.1% to about 99.9% of active ingredient.

The pharmaceutical composition can be processed to prepare a final dosage form—a tablet or a capsule. This can be achieved by compressing the final blend of the combination, optionally together with one or more excipients. The compression can be achieved for example with a rotary tablet press. Tablet of different shapes can be prepared (round, ovaloid, or other suitable shape). The tablet can be coated or uncoated by known techniques to delay disintegration and absorption in the gastrointestinal tract and thereby provide a sustained action over a longer period. If not indicated otherwise, these are prepared in a manner known per se, e.g. by means of mixing, granulating, sugar-coating processes. Formulation for oral use can be presented as hard gelatin capsules wherein the active ingredient is mixed with an inert solid diluent, for example, calcium carbonate, calcium phosphate or cellulose-based excipient, or as soft gelatin capsules wherein the active ingredient is mixed with water or an oil medium, for example, olive oil, liquid paraffin or peanut oil.

The term “effective amount” means the amount of the subject compound that will engender a biological or medical response in a cell, tissue, organ, system, animal or human that is being sought by the researcher, veterinarian, medical doctor or other clinician. The effective dosage of each combination partner agents employed in the combination of the invention may vary depending on the particular compound or pharmaceutical composition employed, the mode of administration, the condition being treated, the severity the condition being treated. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the combination's drugs availability to target sites. This involves a consideration of the distribution, equilibrium and elimination of a drug.

The present disclosure also describes the pharmaceutical combination according to the present disclosure in the form of a “kit of parts” for the combined administration. The combination can refer to either a fixed combination in one dosage unit form, or a kit of parts for the combined administration where (i) ceritinib, or a pharmaceutically acceptable salt thereof, and (ii) EGFR inhibitor, or a pharmaceutically acceptable salt thereof, may be administered independently at the same time or separately within time intervals, especially where these time intervals allow that the combination partners show a cooperative (=joint) effect. The independent formulations or the parts of the formulation, product, or composition, can then, e.g. be administered simultaneously or chronologically staggered, that is at different time points and with equal or different time intervals for any part of the kit of parts. In the combination therapies of the disclosure, the compounds useful according to the disclosure may be manufactured and/or formulated by the same or different manufacturers. Moreover, the combination partners may be brought together into a combination therapy: (i) prior to release of the combination product to physicians (e.g. in the case of a kit comprising ceritinib and the EGFR inhibitor); (ii) by the physician themselves (or under the guidance of a physician) shortly before administration; (iii) in the patient themselves, e.g. during sequential administration of the compound of the disclosure and the other therapeutic agent. In one embodiment the effect of the combination is synergistic.

The therapeutically effective dosage of the combination of the disclosure, or pharmaceutical composition, is dependent on the species of the subject, the body weight, age and individual condition, the disorder or disease or the severity thereof being treated, and can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of the practitioner and each subject's circumstances in view of, e.g., published clinical studies. In general, satisfactory results are indicated to be obtained systemically at daily dosages of from 150 mg to 750 mg of ceritinib orally. In most cases, the daily dose for ceritinib can be between 300 mg and 750 mg. For example gefitinib daily dose can be for example 250 mg orally. Erlotinib can be combined in the usual dose of 150 mg each day, particularly in case when the patient has non-small cell lung cancer. In some cases, 100 mg erlotinib can be given each day. The erlotinib dose may also be adjusted in 50 mg steps.

Cetuximab, for example, is administered once a week with the initial dose of 400 mg cetuximab per m2 body surface area. All subsequent weekly doses are 250 mg cetuximab per m2 each.

For example, Afatinib can administered once daily in dosages from 20 mg to 40 mg.

For example, nimotuzumab can be administered daily in 150 mg/m2.

The term “pharmaceutically acceptable” refers to those compounds, materials, compositions, and/or dosage forms which are suitable for use in contact with the tissues of human beings and animals without excessive toxicity, irritation, allergic response, or other problem or complication, commensurate with a reasonable benefit/risk ratio.

Another aspect of the disclosure is ceritinib for use as a medicine, wherein ceritinib, or a pharmaceutically acceptable salt thereof, is to be administered in combination with an EGFR inhibitor, or a pharmaceutically acceptable salt thereof, for the treatment of an ALK and/or EGFR mediated disease, e.g. cancer.

The term “ALK and/or EGFR mediated disease” refers to a disease in which activity of one or both kinases leads to abnormal activity of the regulatory pathways including overexpression, mutation or relative lack of activity of other regulatory pathways in the cell, e.g. cancer.

### Abbreviations

ALCL: Anaplastic Large Cell Lymphoma
ALK: Anaplastic Lymphoma Kinase.
NSCLC: Non-Small Cell Lung Cancer.
EGFR: Epidermal Growth Factor Receptor.
TKI: Tyrosine Kinase Inhibitor.

The following Examples illustrate the disclosure and provide specific embodiments, however without limiting the scope of the disclosure.

### Example 1. EGFR Ligands Desensitize H2228 Cell Line to Ceritinib Treatment

NCI-H2228 was obtained from ATCC. The cell lines harbored EML4-ALK rearrangements. NCI-H2228 cells were cultured in RPMI-1640 (ATCC Catalog #30-2001) supplemented with 15% FBS.

Secretome screening was performed as described previously (Lito P, Pratilas C A, et al. Cancer Cell 2012; 22: 668-82). In brief, 317 cDNA constructs that represent 220 unique secreted proteins were reverse transfected into HEK293T cells individually and incubated 4 days to allow accumulation of secreted proteins in supernatant. The supernatant was transferred to the assay cells, namely NCI-H2228 cells, followed by addition of ceritinib to a final concentration of 0.5 μM. Cell proliferation was measured using CellTiter-Glo after 96 hours. Cells treated with DMSO in the absence of conditioned media and with ceritinib alone were used as controls.

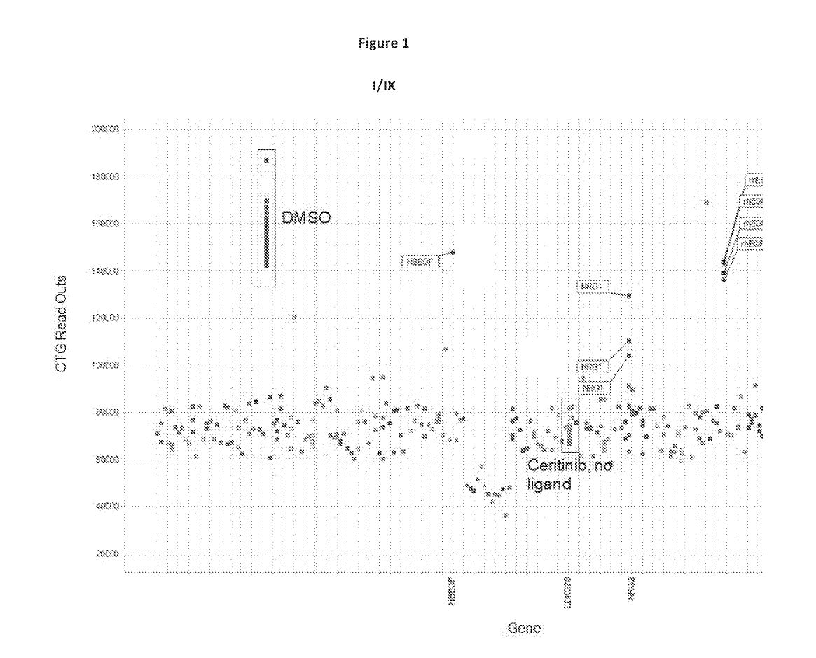

The Cell Titer Glo assay was done with H2228 cell line. The cell line stems from adenocarcinoma; non-small cell lung cancer. Cell Titer Glo assay is a Cell Viability Assay that determined the number of viable cells in culture based on quantitation of the ATP present, which signaled the presence of metabolically active cells. FIG. 1 shows the results.

“Ceritinib no ligand” represents cells that were treated with ceritinib only (absence of any ligand) and represents the impact of ceritinib on cell viability.

“DMSO” represents the basic cell viability (control level) in the presence of a vehicle, but without any compound and all other dots represent ceritinib in the presence of a specific ligand. When cell viability level shifted from the low level of “Ceritinib no ligand” toward DMSO cell viability levels, it means that the particular ligand was able to activate pathway(s) that compensated for the loss of viability due to inhibition of ALK signaling. Ligands for EGFR and ERBB3 clearly overcame the inhibitory effect of ceritinib and could push the cell viability toward the levels observed when cells were treated with DMSO only. EGFR signaling can bypass the ALK inhibition.

The H2228 cell line represents well patient naïve settings—settings where the subject (e.g. patient) has not been pretreated with an ALK inhibitor. Therefore, the experiment indicated that the pharmaceutical combination comprising ceritinib and an EGFR inhibitor would be effective already in naïve patient.

### Example 2. Cell Proliferation Assay of MGH049 and MGH051 Cells

MGH049 and MGH051 were obtained from Massachusetts General Hospital (Friboulet L, Li N, et al. Cancer Discovery 2014; 4: 662-73). These cell lines harbor EML4-ALK rearrangements. MGH049 and MGH051 were cultured in DMEM (ATCC Catalog #30-2002) supplemented with 10% FBS.

To determine the dose-response relationship between the dose of ceritinib and the magnitude of its effect on cell proliferation, 1K MGH049 and 4K MGH051 cells were plated in each well of 384 well-plates, and grown for 24 hours prior to treatment. Cells were then treated with DMSO or ceritinib at concentrations ranging from 4 nM to 1 μM (3-fold dilutions). After 6 days, cell proliferation was measured using the CellTiter-Glo luminescent cell viability assay. Percent inhibition was calculated relative to median DMSO signal.

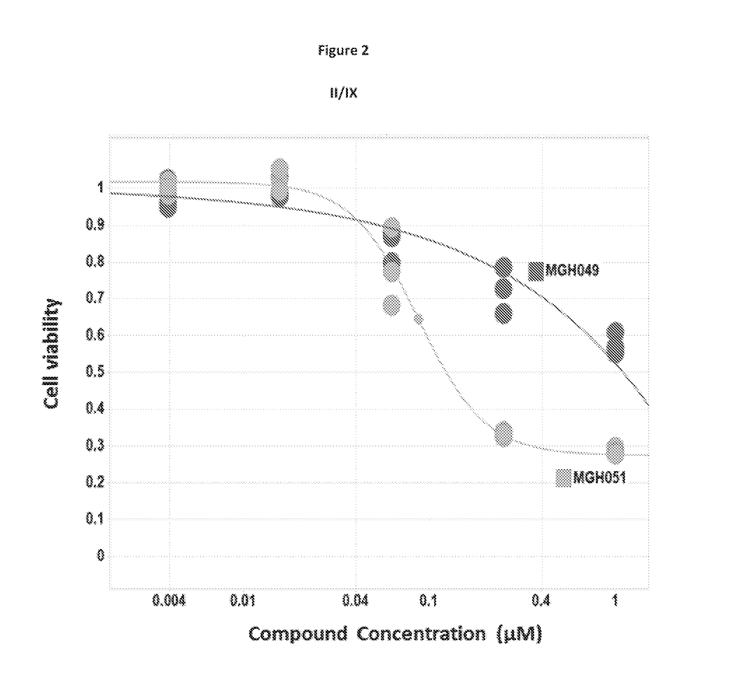

FIG. 2 shows the results of the cell proliferation assay after MGH049 and MGH051 cells were treated with the indicated doses of ceritinib for 6 days. MGH051 is more sensitive to ceritinib treatment than MGH049. The percent inhibition in MGH051 cells is 70% at 0.3 μM ceritinib, while MGH049 is 20%.

### Example 3. Ceritinib in Combination with EGFR Inhibitors Reduce Cell Growth

1K MGH049 and 4K MGH051 cells were plated in each well of 384 well-plates, and treated with escalating doses of lapatinib, erlotinib, gefitinib and Compound A in the following day, in the absence or presence of 0.5 μM ceritinib. At the end of 6 days, inhibition of cell proliferation was assessed using CellTiter-Glo. Three replicate plates were set up for each cell line and drug compound, with or without ceritinib. A representative dose response curve was then calculated by taking the mean across the 3 replicates with and without ceritinib. Proliferation inhibition values were normalized to the measured inhibition value at zero dose of the compound, with and without ceritinib.

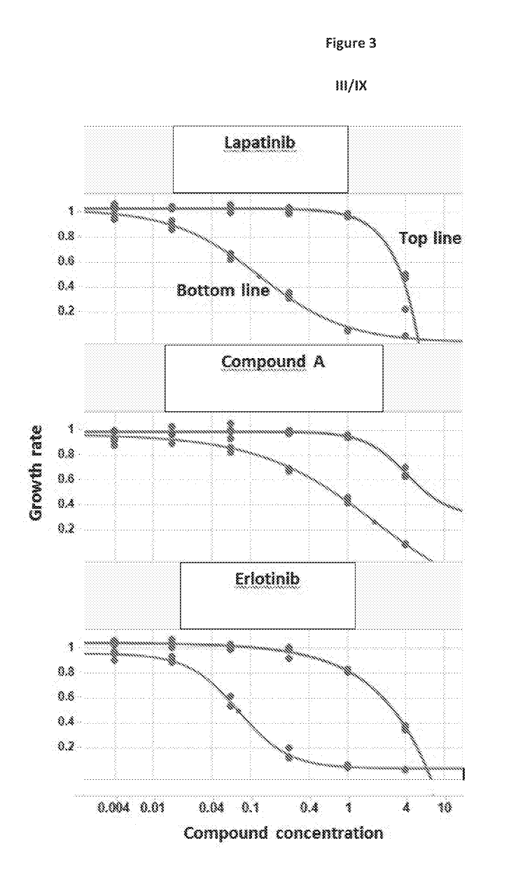

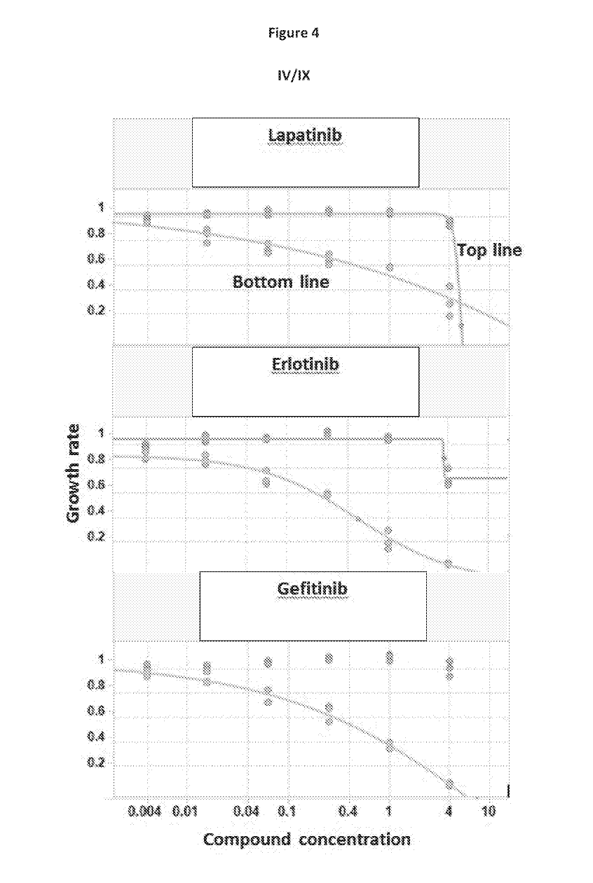

Experiments performed with cell lines MGH049 and MGH051 are depicted on FIG. 3 and FIG. 4, respectively. Cell growth was observed while adding different concentration of each of the EGFR inhibitors (gefitinib, erlotinib, lapatinib or compound A) either singly (top line of the graphs) or each of the EGFR inhibitors in combination with 0.5 μM ceritinib (bottom line on the graphs). From the figures it is clear that the combination synergistically reduced cell growth. Cell line MGH049 was resistant to ceritinib but did not bear any ALK resistance mutations.

The experiment showed that the combination of ceritinib and an EGFR inhibitor was effective even in ALK resistant settings, i.e. where the cells were resistant to the treatment of ceritinib alone. Therefore, the combination provided herein would be useful in cases where patients have been previously treated with an ALK inhibitor (for example post crizotinib, or post ceritinib treatment).

Meaningful data obtained with the combination of ceritinib and gefitinib, erlotinib, lapatinib or compound A, respectively, indicated, that the combination of ceritinib with other EGFR inhibitors is most likely, if not certain, also very advantageous and valuable.

### Example 4. Treatment of ALK Positive NSCLC Cell Lines is Associated with Activation of HER3 (ErbB3)

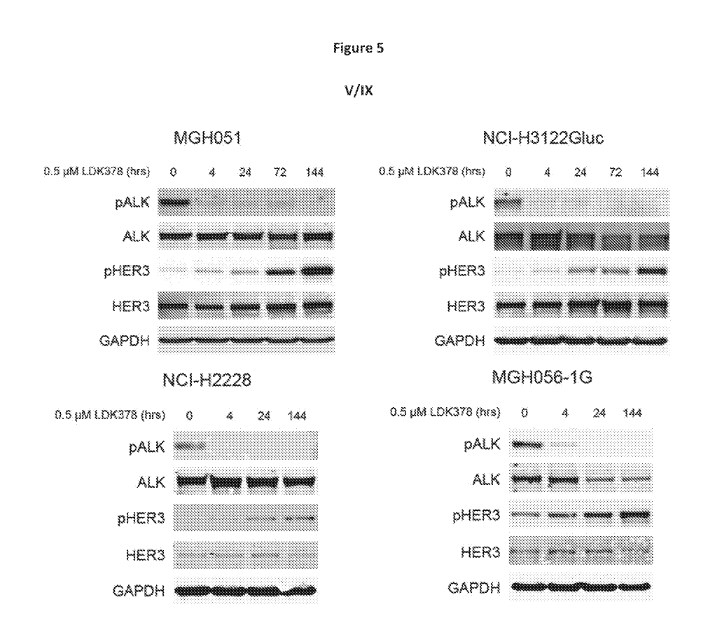

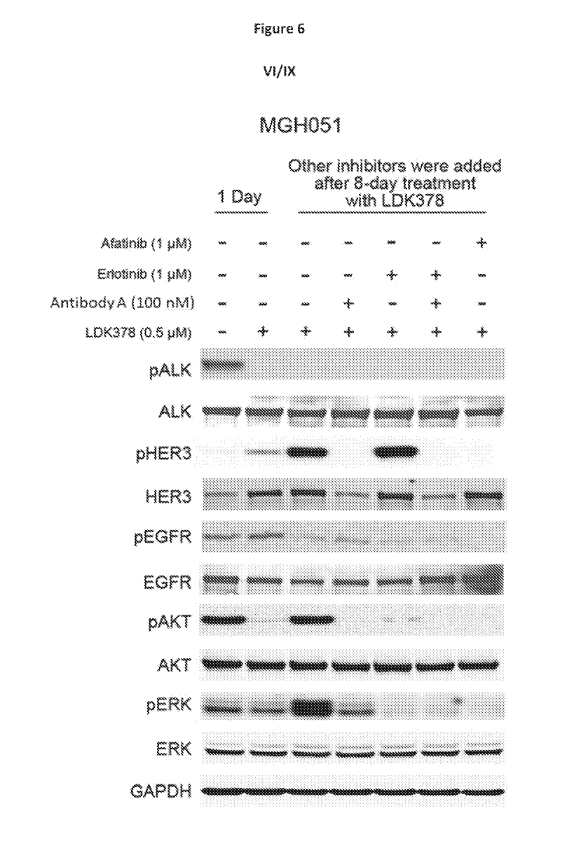

The activated states of ALK and HER3 of multiple NSCLC cell lines that harbor EML4-ALK translocations were assessed in both short-term and long-term cultures. We observed that long-term treatments of 4 ALK positive cell lines with ceritinib (LDK378) led to an induction of HER3 phosphorylation, indicating the activation of HER3 receptors (FIG. 5). In addition, the levels of pHER3 correlated with the duration of the treatments (FIG. 5). Next, we assessed the activation of the downstream signaling of HER3 and the repression of ceritinib-induced HER3 activation using various inhibitors. After initial inhibition of AKT phosphorylation in MGH051, the 8-day treatment of ceritinib resulted in a profound rebound in AKT phosphorylation, accompanying the increased phosphorylation of HER3 (FIG. 6). Addition of Antibody A (MOR10703 from table 1), an anti-HER3 antibody, for the last day of the long-term treatment markedly suppressed the activation of HER3 and the rebound in AKT phosphorylation (FIG. 6). The EGFR inhibitor erlotinib and the pan-HER inhibitor afatinib were also able to suppress the rebound in AKT phosphorylation (FIG. 6), suggesting that the re-activation of the PI3K pathway by HER3 is EGFR-dependent. Furthermore, addition of erlotinib and afatinib for the last day of the treatment led to enhanced inhibition of ERK phosphorylation (FIG. 6). These results suggested that a feedback activation of HER3 is elicited by ALK inhibition, and provided a rational for a therapeutic strategy of combining ALK and HER3 inhibitors or combining ALK and EGFR inhibitors.

### Example 5. EGFR Inhibition Enhances the Inhibition of ALK Positive NSCLC Cell Growth by CERITINIB

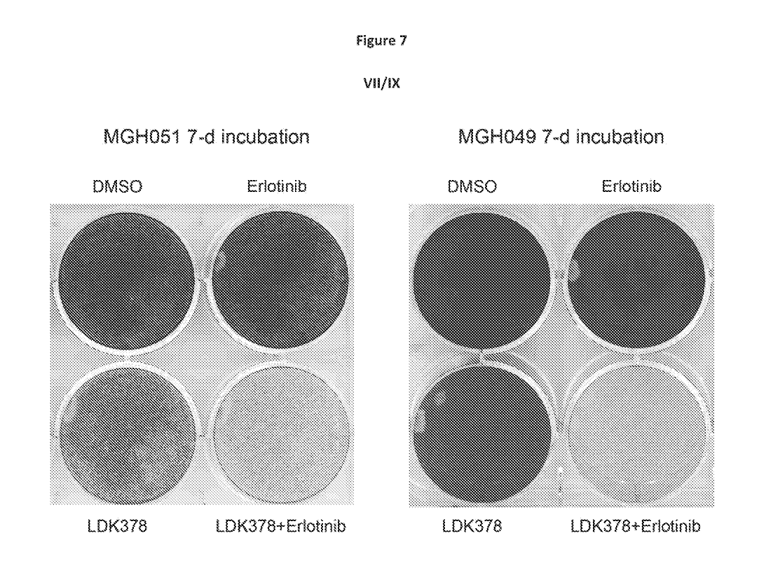

To assess the anti-proliferative effect of ceritinib (LDK378) in combination with the EGFR inhibitor erlotinib, we applied cell colony formation assays using MGH049 and MGH051 cells. Cells were exposed to ceritinib, erlotinib or the combination for 7 days, and stained with crystal violet. As depicted in FIG. 7, ceritinib was partially effective in inhibiting the cell growth of MGHO51, but less effective in MGH049 cells. Ceritinib and erlotinib combination had greater anti-proliferative effect and was more efficacious in killing MGH049 and MGH051 cells than ceritinib alone (FIG. 7). Erlotinib had no effect on cell growth as a single agent, suggesting that these cell lines are ALK-dependent and EGFR signaling functions as a survival pathway or confers resistance to ceritinib (FIG. 7). These results suggested that combination of ALK and EGFR inhibition can either enhance the anti-tumor activity of ceritinib or overcome ceritinib resistance in ALK positive NSCLC.

### Example 6. Efficacy of Ceritinib (LDK378) Alone and in Combination with Cetuximab and/or Antibody A (MOR10703 from Table 1) in H2228 NSCLC Xenograft in Female SCID-Beige Mice

In this experiment female SCID Beige mice bearing H2228 tumors were used. At start, the mice were 8 to 12 weeks of age. First, the mice were inoculated with 4 mm³ H2228 tumor fragments by subcutaneous injection in flank. The fragments were dipped in Matrigel before implanting. When tumors reached an average size of 100-150 mm³ the treatment according to the scheme below commenced. The body weight was measured daily for the first 5 days and then biweekly until end. The tumor was measured biweekly by caliper until end. Ceritinib (LDK378) was administered per os and cetuximab and the Antibody A (Ab A; MOR10703 from table 1) were administered intraperitoneally (ip). Dosing with the compounds ended on study day 32. After this the tumor size was monitored during an observation phase for re-growth.

| | N of | Regimen 1 | | | Regimen 2 | | | Regimen 3 | | |
|---|---|---|---|---|---|---|---|---|---|---|
| # | mice | Agent | mg/kg | Schedule | Agent | mg/kg | Schedule | Agent | mg/kg | Schedule |
| 1^{#} | 8 | vehicle | — | qd x 32 | — | — | — | — | — | — |
| 2 | 7 | LDK378 | 25 | qd x 32 | — | — | — | — | — | — |
| 3 | 7 | LDK378 | 50 | qd x 32 | — | — | — | — | — | — |
| 4 | 7 | LDK378 | 100 | qd x 32 | — | — | — | — | — | — |
| 5 | 7 | cetuximab | 20 | biwk x 5 | — | — | — | — | — | — |
| 6 | 7 | Ab A | 25 | qod x 17 | — | — | — | — | — | — |
| 7 | 8 | Ab A | 25 | qod x 17 | cetuximab | 20 | biwk x 5 | — | — | — |
| 8 | 8 | LDK378 | 25 | qd x 32 | cetuximab | 20 | biwk x 5 | — | — | — |
| 9 | 8 | LDK378 | 50 | qd x 32 | cetuximab | 20 | biwk x 5 | — | — | — |
| 10 | 8 | LDK378 | 25 | qd x 32 | Ab A | 25 | qod x 17 | — | — | — |
| 11 | 8 | LDK378 | 50 | qd x 32 | Ab A | 25 | qod x 17 | — | — | — |
| 12 | 8 | LDK378 | 25 | qd x 32 | cetuximab | 20 | biwk x 5 | Ab A | 25 | qod x 17 |
| 13 | 8 | LDK378 | 50 | qd x 32 | cetuximab | 20 | biwk x 5 | Ab A | 25 | qod x 17 |

^{#}—Control group

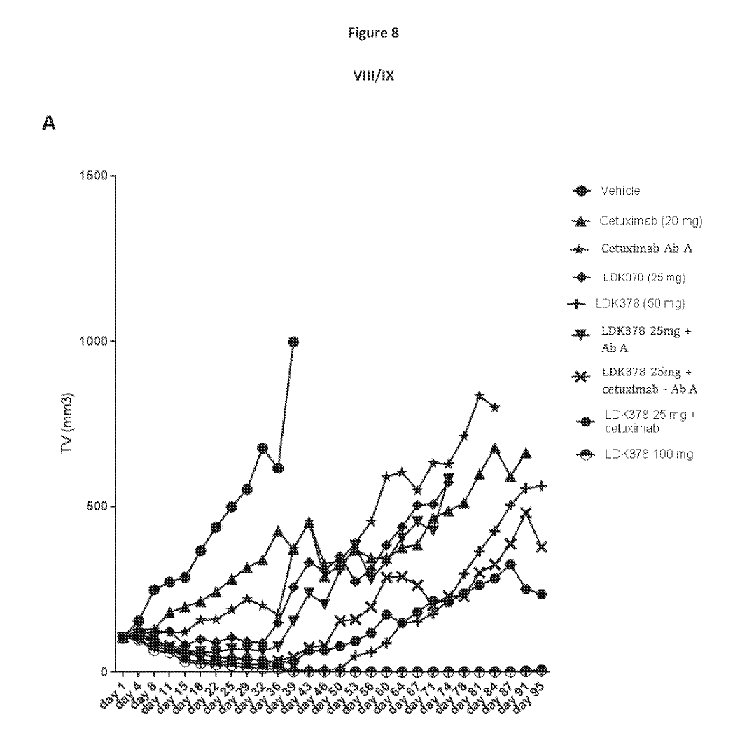

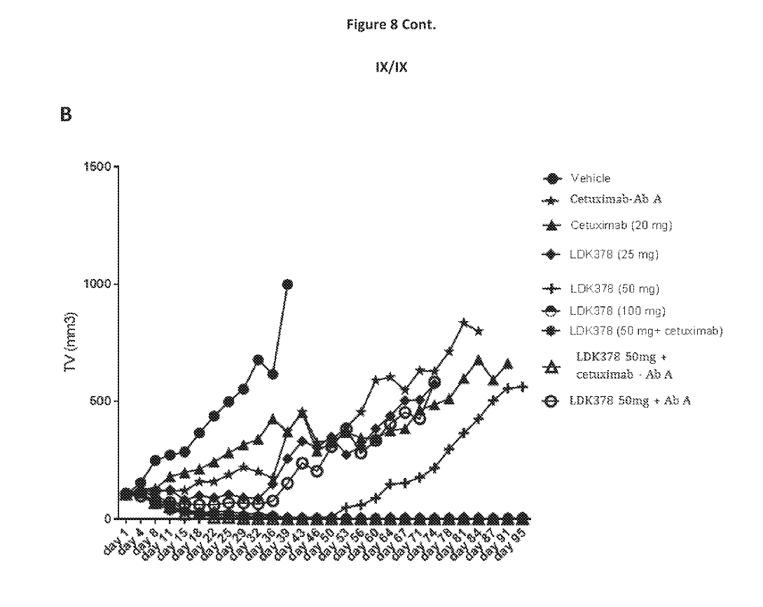

Results are depicted in FIGS. 8A and 8B. The average tumor volume data in the graphs is not complete through the end of the study for all groups. Once a group lost more than 50% of the animals due to death or ethical sacrifice, the average tumor volume for the remaining animals in these groups was no longer graphed. The data showed that the combination of either of the Antibody A or cetuximab significantly improved efficacy of LDK378.

1-43. (canceled)

44. A pharmaceutical combination comprising (i) ceritinib, or a pharmaceutically acceptable salt thereof, and (ii) an EGFR inhibitor, or a pharmaceutically acceptable salt thereof.

45. The pharmaceutical combination according to claim 44, wherein EGFR inhibitor is selected from the group consisting of erlotinib, gefitinib, lapatinib, canetinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, panitumumab, matuzumab, pertuzumab, nimotuzumab, zalutumumab, icotinib, afatinib and cetuximab, and pharmaceutically acceptable salt thereof.

46. The pharmaceutical combination according to claim 44, wherein the EGFR inhibitor is an antibody or fragment thereof comprising the sequences of MOR10703 as defined in Table 1 (Antibody A) or cetuximab, particularly is cetuximab.

47. A method for the treatment of an ALK mediated disease, said method comprising administering an effective amount of a combination of comprising (i) ceritinib, or a pharmaceutically acceptable salt thereof, and (ii) an EGFR inhibitor, or a pharmaceutically acceptable salt thereof, to a subject in need thereof.

48. The method for the treatment according to claim 47, wherein EGFR inhibitor is selected from the group consisting of erlotinib, gefitinib, lapatinib, canertinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, panitumumab, matuzumab, pertuzumab, nimotuzumab, zalutumumab, icotinib, afatinib and cetuximab, and pharmaceutically acceptable salt thereof.

49. The method for the treatment according to claim 48, wherein the EGFR inhibitor is an antibody or fragment thereof comprising the sequences of MOR10703 as defined in Table 1 (Antibody A) or cetuximab, particularly is cetuximab.

50. The method for the treatment of an ALK mediated disease according to claim 47, wherein the disease is cancer.

51. The method for the treatment of an ALK mediated disease according to claim 50, wherein the cancer is NSCLC.

52. The method for the treatment of an ALK mediated disease, according to claim 47, wherein the combination of (i) and (ii) comprises a pharmaceutically acceptable carrier.

53. A method for the treatment of an ALK mediated disease according to claim 47, wherein the EGFR inhibitor is gefitinib.

54. A method for the treatment of an ALK mediated disease according to claim 47, wherein the EGFR inhibitor is erlotinib.

55. A method for the treatment of an ALK mediated disease according to claim 47, wherein the EGFR inhibitor is cetuximab.

56. A method for the treatment of an ALK mediated disease according to claim 47, wherein the EGFR Inhibitor is an isolated antibody or fragment thereof comprising a heavy chain CDR3 selected from the group consisting of SEQ ID NO: 4, SEQ ID NO: 10, SEQ ID NO: 22, SEQ ID NO: 28, SEQ ID NO: 40, SEQ ID NO: 46, SEQ ID NO: 58, SEQ ID NO: 64, SEQ ID NO: 76, SEQ ID NO: 82, SEQ ID NO: 94, SEQ ID NO: 100, SEQ ID NO: 112, SEQ ID NO: 118, SEQ ID NO: 130, SEQ ID NO: 136, SEQ ID NO: 148, SEQ ID NO: 166, SEQ ID NO: 184, SEQ ID NO: 202, SEQ ID NO: 220, SEQ ID NO: 238, SEQ ID NO: 256, SEQ ID NO: 274, SEQ ID NO: 292, SEQ ID NO: 310, SEQ ID NO: 328, SEQ ID NO: 346, and SEQ ID NO: 364.

57. A method for the treatment of an ALK mediated disease according to claim 47, wherein the EGFR Inhibitor is an isolated antibody or fragment thereof that comprises a heavy chain variable region CDR1 of SEQ ID NO: 128; CDR2 of SEQ ID NO: 129; CDR3 of SEQ ID NO: 130; and a light chain variable region CDR1 of SEQ ID NO: 131; CDR2 of SEQ ID NO: 132; and CDR3 of SEQ ID NO: 133.

58. A method for the treatment of an ALK mediated disease according to claim 47, wherein the EGFR inhibitor is a combination of an isolated antibody or fragment thereof that comprises a heavy chain variable region CDR1 of SEQ ID NO: 128; CDR2 of SEQ ID NO: 129; CDR3 of SEQ ID NO: 130; and a light chain variable region CDR1 of SEQ ID NO: 131; CDR2 of SEQ ID NO: 132; and CDR3 of SEQ ID NO: 133 and cetuximab.

59. A method for the treatment of an ALK mediated disease according to claim 47, wherein the disease is EGFR wt.

60. A method for the treatment of an ALK mediated disease according to claim 47, wherein the disease comprises T790M EGFR.

61. A method for the treatment of an ALK mediated disease according to claim 47, wherein ceritinib and EGFR inhibitor are administered to an ALK-naïve patient.

62. A method for the treatment of an ALK mediated disease according to claim 47, wherein ceritinib and EGFR inhibitor are administered to an patient that has been pretreated with an ALK Inhibitor.

63. A method for the treatment of an ALK mediated disease according to claim 47, wherein ceritinib and EGFR inhibitor are administered to an patient that has been pretreated with ceritinib.
